(19) United States
(12) Patent Application Publication
(10) Pub. No.: US 2025/0360036 A1
(43) Pub. Date: Nov. 27, 2025

(54) PRESSURE-MITIGATION APPARATUSES DESIGNED WITH CHAMBER MODULARITY AND APPROACHES TO DYNAMICALLY USING THE SAME TO ALLEVIATE PRESSURE

(71) Applicant: TurnCare, Inc., Palo Alto, CA (US)

(72) Inventor: Rafael Paolo Squitieri, Wilton, CT (US)

(21) Appl. No.: 19/293,415

(22) Filed: Aug. 7, 2025

Related U.S. Application Data
(63) Continuation of application No. PCT/US2024/012826, filed on Jan. 24, 2024.
(60) Provisional application No. 63/485,830, filed on Feb. 17, 2023.

(51) Int. Cl. A61G 7/057

(52) U.S. Cl. CPC A61G 7/05776

(57) ABSTRACT

Introduced here are pressure-mitigation devices having improved adaptability or customizability to specific patients. An example pressure-mitigation device features modularity and dynamic usage of a number of individual chamber devices that can be assembled for a given body size, a given environment or substrate, and/or the like. Each modular chamber device includes at least one inflatable chamber and is configured for independent inflation and/or deflation of its at least one inflatable chamber. A set of modular chamber devices arranged (and attached) together can be operated in concert to provide a pressure-mitigation treatment for a body disposed atop and across the set of modular chamber devices.

### CROSS-REFERENCE TO RELATED APPLICATIONS

This application is a continuation of International Application No. PCT/US2024/012826, titled “PRESSURE-MITIGATION APPARATUSES DESIGNED WITH CHAMBER MODULARITY AND APPROACHES TO DYNAMICALLY USING THE SAME TO ALLEVIATE PRESSURE” and filed Jan. 24, 2024, which claims priority to U.S. Provisional Application No. 63/485,830, titled “PRESSURE-MITIGATION APPARATUSES DESIGNED WITH CHAMBER MODULARITY AND APPROACHES TO DYNAMICALLY USING THE SAME TO ALLEVIATE PRESSURE” and filed on Feb. 17, 2023, which are incorporated herein by reference in their entireties.

### TECHNICAL FIELD

Various embodiments concern pressure-mitigation apparatuses able to alleviate pressure applied on a body, such as a human body.

### BACKGROUND

Pressure injuries-sometimes referred to as “decubitus ulcers,” “pressure ulcers,” “pressure sores,” or “bedsores”—may occur as a result of steady pressure being applied in one location along the surface of the human body for a prolonged period of time. Regions with bony prominences are especially susceptible to pressure injuries. Pressure injuries are most common in individuals who are completely immobilized (e.g., on an operating table, bed, or chair) or have impaired mobility. These individuals may be older, malnourished, or incontinent, all factors that predispose the human body to formation of pressure injuries.

These individuals are often not ambulatory, so they sit or lie for prolonged periods of time in the same position. Moreover, these individuals may be unable to reposition themselves to alleviate pressure. Consequently, pressure on the skin and underlying soft tissue may eventually result in inadequate blood flow to the area, a condition referred to as “ischemia,” thereby resulting in damage to the skin or underlying soft tissue. Pressure injuries can take the form of a superficial injury to the skin or a deeper ulcer that exposes the underlying tissues and places the individual at risk for infection. The resulting infection may worsen, leading to sepsis or even death in some cases.

There are various technologies on the market that profess to prevent pressure injuries. However, these conventional technologies have many deficiencies. For instance, these conventional technologies are unable to control the spatial relationship between a human body and a support surface (or simply “surface”) that applies pressure to the human body. Conventional technologies are also unable to effectively coordinate the use of multiple surfaces that apply pressure to various parts of the human body. Consequently, individuals that use these conventional technologies have to operate multiple devices that control multiple surfaces, with the outcome being that they may still develop pressure injuries or suffer from related complications.

### BRIEF DESCRIPTION OF THE DRAWINGS

FIGS. 1A-1B are top and bottom views, respectively, of a pressure-mitigation device able to relieve the pressure on an anatomical region applied by the surface of an elongated object in accordance with embodiments of the present technology.

FIGS. 2A and 2B are top and bottom views, respectively, of a pressure-mitigation device configured in accordance with embodiments of the present technology.

FIG. 3 is a top view of a pressure-mitigation device for relieving pressure on an anatomical region of a patient in accordance with embodiments of the present technology.

FIG. 4 is a top view of a pressure-mitigation device for relieving pressure on an anatomical region applied by an elongated object in accordance with embodiments of the present technology.

FIG. 5 is a partially schematic top view of a pressure-mitigation device illustrating how a pressure gradient can be created by varying pressure distributions to avoid ischemia in a mobility-impaired patient in accordance with embodiments of the present technology.

FIG. 6A is a partially schematic side view of a pressure-mitigation device for relieving pressure on a specific anatomical region by deflating one or more chambers in accordance with embodiments of the present technology.

FIG. 6B is a partially schematic side view of a pressure-mitigation device for relieving pressure on a specific anatomical region by inflating one or more chambers in accordance with embodiments of the present technology.

FIG. 7 illustrates a modular application of multiple pressure-mitigation devices in which the multiple pressure-mitigation devices are connected to one another to form an aggregate pressure-mitigation device, in accordance with embodiments of the present technology.

FIGS. 8A-8E illustrate example modular chamber devices that are configured to be selectively combined and connected to other modular chamber devices in a geometric arrangement to form an aggregate pressure-mitigation device, in accordance with embodiments of the present technology.

FIG. 8F illustrates an aggregate pressure-mitigation device with no intervening tubing between its modular chamber devices, in accordance with embodiments of the present technology.

FIG. 9A illustrates an example of modular controller-pump assemblies included in modular chamber devices of an aggregate pressure-mitigation device, in accordance with embodiments of the present technology.

FIG. 9B illustrates examples of inter-controller communications for pressure-mitigation devices and sets thereof, in accordance with embodiments of the present technology.

FIG. 10 is a flow diagram of alleviating pressure experienced by a patient using an aggregate pressure-mitigation device in accordance with embodiments of the present technology.

FIGS. 11A-11C are isometric, front, and back views, respectively, of a controller device (also referred to as a “controller”) that is configured for controlling inflation and/or deflation of one or more independent chamber devices and/or chambers of a pressure-mitigation device in accordance with embodiments of the present technology.

FIG. 12 illustrates an example of a controller in accordance with embodiments of the present technology.

FIG. 13 is a block diagram illustrating an example of a processing system in which at least some operations described herein can be implemented.

Various features of the technologies described herein will become more apparent to those skilled in the art from a study of the Detailed Description in conjunction with the drawings. Embodiments are illustrated by way of example and not limitation in the drawings. While the drawings depict various embodiments for the purpose of illustration, those skilled in the art will recognize that alternative embodiments may be employed without departing from the principles of the technologies. Accordingly, while specific embodiments are shown in the drawings, the technology is amenable to various modifications.

### DETAILED DESCRIPTION

The term “pressure injury” refers to a localized region of damage to the skin and/or underlying tissue that results from contact pressure (or simply “pressure”) on the corresponding anatomical region of the human body. Pressure injuries will often form over bony prominences, such as the skin and soft tissue overlying the sacrum, coccyx, heels, or hips. However, other sites may also be affected. For instance, pressure injuries may form on the elbows, knees, ankles, shoulders, abdomen, back, or cranium. Pressure injuries may develop when pressure is applied to the blood vessels in soft tissue in such a manner that blood flow to the soft tissue is at least partially obstructed (e.g., due to the pressure exceeding the capillary filling pressure), and ischemia results at the site when such obstruction occurs for an extended duration. Accordingly, pressure injuries are normally observed on individuals who are mobility impaired, immobilized, or sedentary for prolonged periods of times.

Once pressure injuries have formed, the healing process is normally slow. When pressure is relieved from the site of a pressure injury, the body will rush blood (with proinflammatory mediators) to that region to perfuse the area with blood. The sudden reperfusion of the damaged (and previously ischemic) region has been shown to cause an inflammatory response, brought on by the proinflammatory mediators, that can actually worsen the pressure injury (and thus prolong recovery). Moreover, in some cases, the proinflammatory mediators may spread through the blood stream beyond the site of the pressure injury to cause a systematic inflammatory response (also referred to as a “secondary inflammatory response”). Secondary inflammatory responses caused by proinflammatory mediators have been shown to exacerbate existing conditions and/or trigger new conditions, thereby slowing recovery. Recovery can also be prolonged by factors that are frequently associated with individuals who are prone to pressure injuries, such as old age, immobility, preexisting medical conditions (e.g., arteriosclerosis, diabetes, or infection), smoking, and medications (e.g., anti-inflammatory drugs). Inhibiting the formation of pressure injuries (and reducing the prevalence of proinflammatory mediators) can enhance and expedite many treatment processes, especially for those individuals whose mobility is impaired during treatment.

Introduced here, therefore, are pressure-mitigation devices able to mitigate the pressure applied to a human body by the surface of an object (also referred to as a “structure”). A controller device (or simply “controller”) can be fluidically coupled to a pressure-mitigation device (also referred to as a “pressure-mitigation apparatus” or a “pressure-mitigation pad”) that includes a series of selectively inflatable chambers (also referred to as “cells” or “compartments”). When a pressure-mitigation device is placed between a human body and a surface, the controller can continuously, intelligently, and autonomously circulate fluid through the chambers of the pressure-mitigation device. Normally, the controller circulates air through the chambers of the pressure-mitigation device, though the controller could circulate another fluid, such as water or gel, through the chambers of the pressure-mitigation device. As further discussed below, the controller may cause the chambers to be selectively inflated, deflated, or any combination thereof.

The present disclosure is directed to pressure-mitigation devices configured with chamber modularity and adaptable uses thereof for different body sizes and environments. An example pressure-mitigation device may include separate or individual modular chamber devices that are arranged, intertwined, interlaced, and/or connected together to form a geometric arrangement of inflatable chambers for the pressure-mitigation device. Different numbers of individual modular chamber devices and different respective shapes thereof may be used to form a particular geometric arrangement of inflatable chambers specific to a particular type of pressure-mitigation treatment (e.g., a treatment for a sacral region of a body, a treatment for a thoracic region of the body, a treatment for a cranial region of the body), specific to a particular subject (e.g., a neonatal subject, an adult subject), and/or specific to a treatment environment or substrate (e.g., a mattress, a chair, a wheelchair). Therefore, for example, a pressure-mitigation device can be adapted, re-arranged (with respect to its inflatable chambers), and re-used, through selective inclusion of specific individual modular chamber devices, for improved applicability and usage.

Example embodiments provide modular chamber devices composed of an inflatable chamber configured in a geometric shape that can fit adjacent and/or flush to other modular chamber devices configured in the same shape or other shapes. In some examples, a modular chamber device includes interconnection mechanisms, such as snaps, buttons, magnets, and others, via which the modular chamber device can be detachably connected with other modular chamber devices to form an aggregate pressure-mitigation device with multiple inflatable chambers in a particular geometric arrangement for providing a pressure-mitigation treatment. In some embodiments, a modular chamber device includes more than one inflatable chamber (e.g., two chambers, three chambers, four chambers) and can be connected with other modular chamber devices to increase a total aggregate number of inflatable chambers that are used to provide a pressure-mitigation treatment.

According to example embodiments, a given modular chamber device is configured to be individually operable to inflate and deflate its inflatable chamber(s). In particular, in example embodiments, a modular chamber device includes a respective controller and mechanisms for inflating and deflating the inflatable chamber(s). When multiple modular chamber devices are connected and aggregated to form a pressure-mitigation device, respective controllers of the multiple modular chamber devices may cooperate and inter-communicate such that the pressure-mitigation device provides a pressure-mitigation treatment via coordinated inflation and deflation of multiple inflatable chambers. Cooperation and coordination of the respective controllers of multiple modular chamber devices may occur via communication (e.g., wireless communication or wired communication) between the respective controllers. For example, a controller communicates, to other controllers, an inflation state of a corresponding inflatable chamber, such that the other controllers can appropriately control their corresponding inflatable chambers. As another example, the controllers synchronize on a timepoint or time-based pattern for periodically inflating and deflating their respective chambers. Communication between controllers of modular chamber devices can occur at an initial synchronization point, occur continuously or periodically, occur on an ad hoc basis in response to certain events, and/or the like. In an example, the controllers maintain open communication and relay information on real-time statuses, such as inflation states or cycle points. In another example, the controllers undergo an initial communication or synchronization process to establish a share timing, after which inter-controller communication does not occur, or only occurs on certain events (e.g., detection of a fault or error, addition of a new controller, removal of a controller, periodic resynchronization points). Various techniques for coordination, cooperation, and synchronization between multiple modular chamber devices in an aggregate pressure-mitigation device are discussed herein.

Embodiments may be described with reference to particular anatomical regions, treatment regimens, environments, etc. However, those skilled in the art will recognize that the features are similarly applicable to other anatomical regions, treatment regimens, environments, etc. As an example, embodiments may be described in the context of a pressure-mitigation device that is positioned adjacent to an anterior anatomical region of an individual oriented in the prone position. However, aspects of those embodiments may apply to a pressure-mitigation device that is positioned adjacent to a posterior anatomical region of an individual oriented in the supine position.

While embodiments may be described in the context of machine-readable instructions, aspects of the technology can be implemented via hardware, firmware, or software. As an example, a controller may not only execute instructions for determining an appropriate rate at which to permit fluid (e.g., air) flow into the inflatable chamber of a pressure-mitigation device but may also be responsible for facilitating communication with other computing devices. For example, the controller may be able to communicate with a mobile device that is associated with the individual or a caregiver, or the controller may be able to communicate with a computer server of a network-accessible server system.

### Terminology

References in this description to “an embodiment” or “one embodiment” means that the feature, function, structure, or characteristic being described is included in at least one embodiment of the technology. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” and variants thereof are intended to include any connection or coupling between two or more elements, either direct or indirect. The connection/coupling can be physical, logical, or a combination thereof. For example, objects may be electrically or communicatively coupled to one another despite not sharing a physical connection.

The term “module” may refer to software components, firmware components, or hardware components. Modules are typically functional components that generate one or more outputs based on one or more inputs. As an example, a computer program may include multiple modules responsible for completing different tasks or a single module responsible for completing all tasks.

When used in reference to a list of multiple items, the term “or” is intended to cover all of the following interpretations: any of the items in the list, all of the items in the list, and any combination of items in the list.

The sequences of steps performed in any of the processes described here are exemplary. However, unless contrary to physical possibility, the steps may be performed in various sequences and combinations. For example, steps could be added to, or removed from, the processes described here. Similarly, steps could be replaced or reordered. Thus, descriptions of any processes are intended to be open ended.

### Overview of Pressure-Mitigation Devices

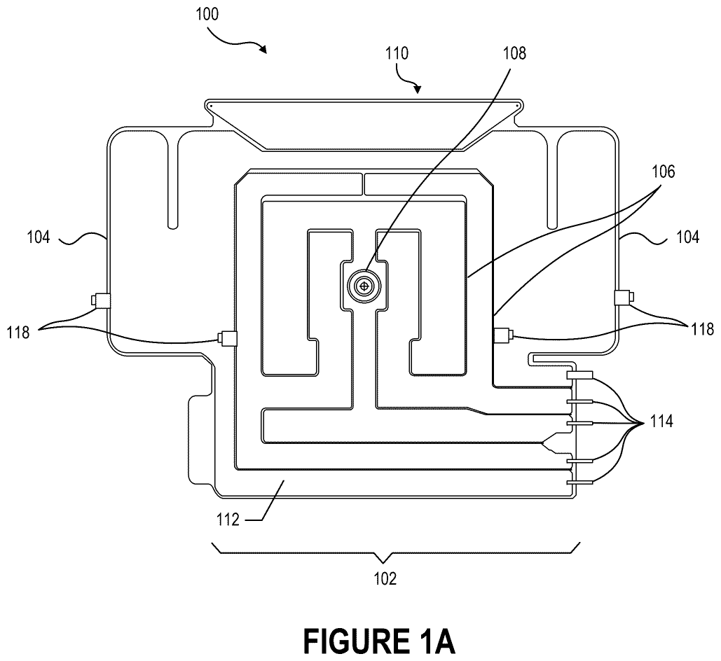

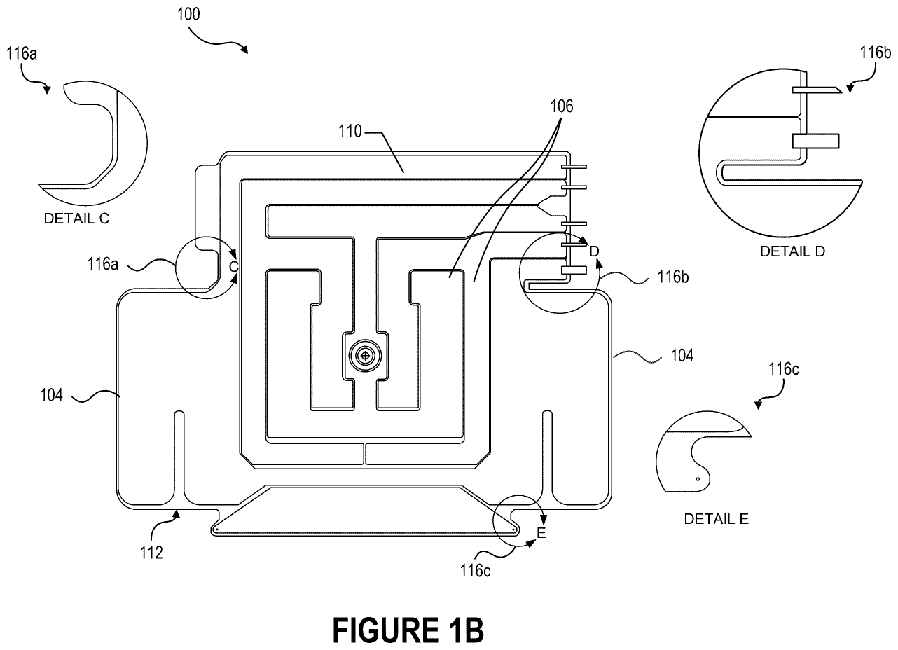

A pressure-mitigation apparatus includes a plurality of chambers or compartments that can be individually controlled to vary the pressure in each chamber and/or a subset of the chambers. When placed between a human body and a support surface, the pressure-mitigation apparatus can vary the pressure on an anatomical region by controllably inflating one or more chambers, deflating one or more chambers, or any combination thereof. Several examples of pressure-mitigation apparatuses are described below with respect to FIGS. 1A-3. Unless otherwise noted, any features described with respect to one embodiment are equally applicable to the other embodiments. Some features have only been described with respect to a single embodiment of the pressure-mitigation apparatus for the purpose of simplifying the present disclosure.

FIGS. 1A-1B are top and bottom views, respectively, of an example of a pressure-mitigation device 100, able to relieve the pressure on an anatomical region applied by the surface of an elongated object in accordance with embodiments of the present technology. While the pressure-mitigation device 100 may be described in the context of elongated objects, such as mattresses, stretchers, operating tables, and procedure tables, the pressure-mitigation device 100 could be deployed on non-elongated objects.

In some embodiments, the pressure-mitigation device 100 is secured to a support surface or substrate (e.g., a mattress, a cushion, a pad) using an attachment apparatus. In other embodiments, the pressure-mitigation device 100 is placed in direct contact with the surface without any attachment apparatus therebetween. For example, the pressure-mitigation device 100 may have a tacky substance deposited along at least a portion of its outer surface that allows it to temporarily adhere to the surface. Examples of tacky substances include latex, urethane, and silicone rubber. However, as discussed, these techniques involve additional equipment or materials, and reliability of such techniques are improved upon in embodiments disclosed herein.

As shown in FIG. 1A, the pressure-mitigation device 100 can include a central portion 102 (also referred to as a “contact portion”) that is positioned alongside at least one side support 104. Here, a pair of side supports 104 are arranged on opposing sides of the central portion 102. However, some embodiments of the pressure-mitigation device 100 do not include any side supports. For example, the side support(s) 104 may be omitted when the individual is medically immobilized (e.g., under anesthesia, in a medically induced coma, etc.) and/or physically restrained by underlying object (e.g., by rails along the side of a bed, armrests along the side of a chair, etc.) or some other structure (e.g., physical restraints, casts, etc.).

The pressure-mitigation device 100 includes a series of chambers 106 whose pressure can be individually varied. In some embodiments, the series of chambers 106 are arranged in a geometric pattern designed to relieve pressure on specific anatomical region(s) of a human body. As noted above, when placed between the human body and a surface, the pressure-mitigation device 100 can vary the pressure on these specific anatomical region(s) by controllably inflating and/or deflating chamber(s).

In some embodiments, the series of chambers 106 are arranged such that pressure on a given anatomical region is mitigated when the given anatomical region is oriented over a target region 108 of the geometric pattern. As shown in FIGS. 1A-1B, the target region 108 may be representative of a central point of the pressure-mitigation device 100 to appropriately position the anatomy of the human body with respect to the pressure-mitigation device 100. For example, the target region 108 may correspond to an epicenter of the geometric pattern. However, the target region 108 may not necessarily be the central point of the pressure-mitigation device 100, particularly if the series of chambers 106 are positioned in a non-symmetric arrangement. The target region 108 may be visibly marked so that an individual can readily align the target region 108 with a corresponding anatomical region of the human body to be positioned thereon. Thus, the pressure-mitigation device 100 may include a visual element representative of the target region 108 to facilitate alignment with the corresponding anatomical region of the human body. The individual could be a physician, nurse, caregiver, or the patient.

The pressure-mitigation device 100 can include a first portion 110 (also referred to as a “first layer” or “bottom layer”) designed to face a surface and a second portion 112 (also referred to as a “second layer” or “top layer”) designed to face the human body supported by the surface. In some embodiments, the pressure-mitigation device 100 is deployed such that the first portion 110 is directly adjacent to the surface. For example, the first portion 110 may have a tacky substance deposited along at least a portion of its exterior surface that facilitates temporarily adhesion to the support surface. In other embodiments, the pressure-mitigation device 100 is deployed such that the first portion 110 is directly adjacent to an attachment apparatus designed to help secure the pressure-mitigation device 100 to the support surface. The pressure-mitigation device 100 may be constructed of various materials, and the material(s) used in the construction of each component of the pressure-mitigation device 100 may be chosen based on the nature of the body contact, if any, to be experienced by the component. For example, because the second portion 112 will often be in direct contact with the skin, it may be comprised of a soft fabric or a breathable fabric (e.g., comprised of moisture-wicking materials or quick-drying materials, or having perforations). In some embodiments, an impervious lining (e.g., comprised of polyurethane) is secured to the inside of the second portion 112 to inhibit fluid (e.g., sweat) from entering the series of chambers 106. As another example, if the pressure-mitigation device 100 is designed for deployment beneath a cover (e.g., a bed sheet), then the second portion 112 may be comprised of a flexible, liquid-impervious material, such as polyurethane, polypropylene, silicone, or rubber. The first portion 110 may also be comprised of a flexible, liquid-impervious material.

Generally, the first and second portions 110, 112 are selected and/or designed such that the pressure-mitigation device 100 is readily cleanable. However, the specific materials that are used may vary depending on the environment in which the pressure-mitigation device 100 is to be deployed. Assume, for example, that the pressure-mitigation device 100 is intended to be deployed in a hospital environment. In such a scenario, the first and second portions 110, 112 may be readily cleanable with a cleaning agent (e.g., bleach) or a cleaning procedure (e.g., sterilization). Because the pressure-mitigation device 100 will remain in the hospital environment under the care of knowledgeable persons, the first and second portions 110, 112 could be comprised of materials that may degrade quickly if not properly cared for. Examples of such materials include high-performance fabric, upholstery, vinyl, and other suitable textiles. If the pressure-mitigation device 100 is instead intended to be deployed in a home environment, the first and second portions 110, 112 may be comprised of materials that can be readily cleaned by persons without extensive experience. For example, the first portion 110 and/or the second portion 112 may be comprised of a vinyl that is easy to clean with commonly available cleaning agents (e.g., bleach, liquid dish soap, all-purpose cleaners). Regardless of the environment, the first and second portions 110, 112 may contain antimicrobial additives, antifungal additives, flame-retardant additives, and the like.

The series of chambers 106 may be formed via interconnections between the first and second portions 110, 112. For example, the first and second portions 110, 112 may be bound directly to one another, or the first and second portions 110, 112 may be bound to one another via one or more intermediary layers. In the embodiment illustrated in FIGS. 1A-1B, the pressure-mitigation device 100 includes an “M-shaped” chamber intertwined with two “C-shaped” chambers that face one another. Such an arrangement has been shown to effectively mitigate the pressure applied to the sacral region of a human body in the supine position by a support surface when the pressure in these chambers is alternated. The series of chambers 106 may be arranged differently if the pressure-mitigation device 100 is designed for an anatomical region other than the sacral region, or if the pressure-mitigation device 100 is to be used to support a human body in a non-supine position (e.g., a prone position or sitting position). Generally, the geometric pattern of chambers 106 is designed based on the internal anatomy (e.g., the muscles, bones, and vasculature) of the anatomical region on which pressure is to be relieved.

The person using the pressure-mitigation device 100 and/or the caregiver (e.g., a nurse, physician, family member, etc.) may be responsible for actively orienting the anatomical region of the human body lengthwise over the target region 108 of the geometric pattern. If the pressure-mitigation device 100 includes one or more side supports 104, the side support(s) 104 may actively orient or guide the anatomical region of the human body laterally over the target region 108 of the geometric pattern. In some embodiments the side support(s) 104 are inflatable, while in other embodiments the side support(s) 104 are permanent structures that protrude from one or both lateral sides of the pressure-mitigation device 100. For example, at least a portion of each side support may be stuffed with cotton, latex, polyurethane foam, or any combination thereof.

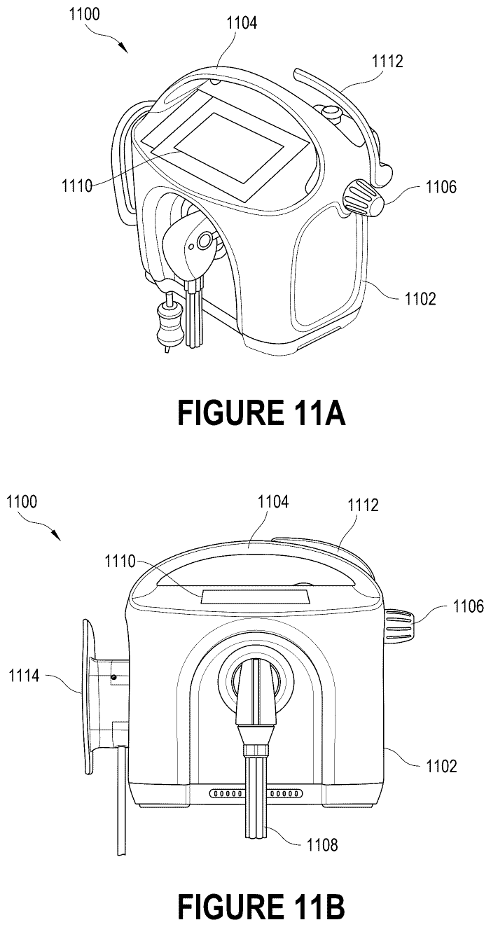

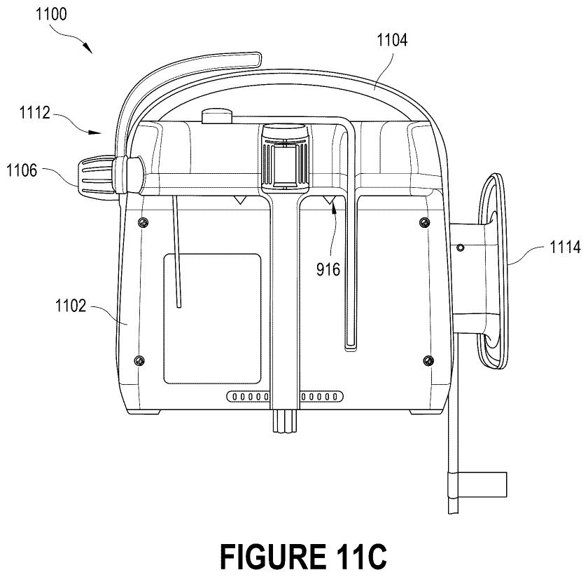

As further described below (e.g., with respect to FIGS. 11A-11C), a controller can separately or independently control the pressure in each chamber (as well as the side supports 104, if included) by providing a discrete airflow via one or more corresponding valves 114. In some embodiments, the valves 114 are permanently secured to the pressure-mitigation device 100 and designed to interface with tubing that can be readily detached (e.g., for easier transport, storage, etc.). Here, the pressure-mitigation device 100 includes five valves 114. Three valves are fluidically coupled to the series of chambers 106, and two valves are fluidically coupled to the side supports 104. Other embodiments of the pressure-mitigation device 100 may include more than five valves or less than five valves. For example, the pressure-mitigation device 100 may be designed such that a pair of side supports 104 are pressurized via a single airflow received via a single valve.

In some embodiments, the pressure-mitigation device 100 includes one or more design features 116a-c designed to facilitate securement of the pressure-mitigation device 100 to the surface of an object and/or an attachment apparatus. As illustrated in FIG. 1B, for example, the pressure-mitigation device 100 may include three design features 116a-c, each of which can be aligned with a corresponding structural feature that is accessible along the surface of the object or the attachment apparatus. For example, each design feature 116a-c may be designed to at least partially envelope a structural feature that protrudes upward. One example of such a structural feature is a rail that extends along the side of a bed. The design feature(s) 116a-c may also facilitate proper alignment of the pressure-mitigation device 100 with the surface of the object or the attachment apparatus.

While not shown in FIGS. 1A-1B, one or more release valves (also referred to as “discharge valves”) may be located along the periphery of the pressure-mitigation device 100 to allow for quick discharge of the fluid stored therein. Normally, the release valve(s) are located along the longitudinal sides to ensure that the release valve(s) are not located beneath a human body situated on the pressure-mitigation device 100. Release valve(s) may allow discharge of fluid from the side supports 104 and/or the series of chambers 106. In some embodiments, fluid is separately or collectively dischargeable from the side supports 104 (e.g., where each side support has at least one release valve). Such a design is desirable in some scenarios because fluid can quickly be discharged from the side supports 104, which allows the human body situated on the pressure-mitigation device 100 to be accessed (e.g., in the case of a medical emergency). In other embodiments, fluid is only collectively dischargeable from the side supports 104. This approach to “dually deflating” the side supports 104 may be taken if release valve(s) are connected to only one side support, though both side supports are fluidically coupled to one another. The release valve(s) may be manually or electrically actuated. For example, the release valve(s) may be manually actuated by pressing a mechanical button (also referred to as a “strike button”) that, when pressed, allows fluid to flow out of the corresponding chamber or side support. In embodiments where the fluid is air, the air may be permitted to flow into the ambient environment. In embodiments where the fluid is water or gel, the fluid may be directed into a container (e.g., from which the fluid can then be rerouted through the controller as further discussed below). As another example, the release valve(s) may be electronically actuated by interacting with a switch assembly (e.g., located along the exterior surface of the pressure-mitigation device 100), a controller, or another computing device (e.g., a mobile phone or wearable electronic device) that is communicatively connected to the pressure-mitigation device 100.

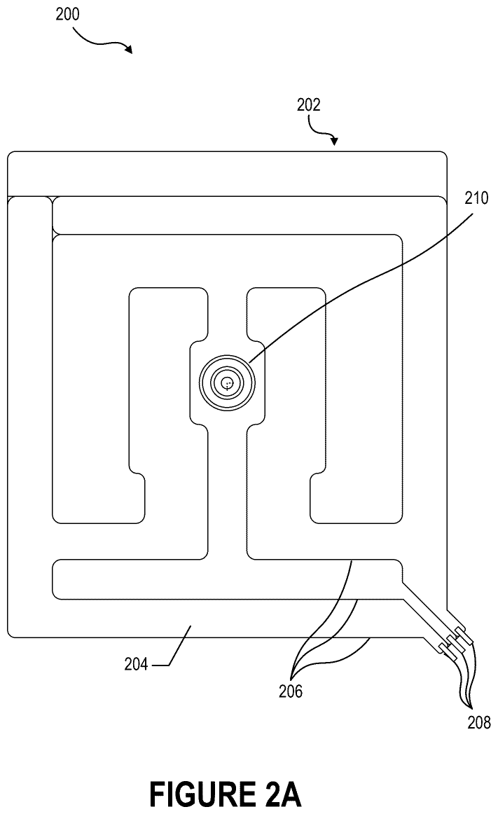

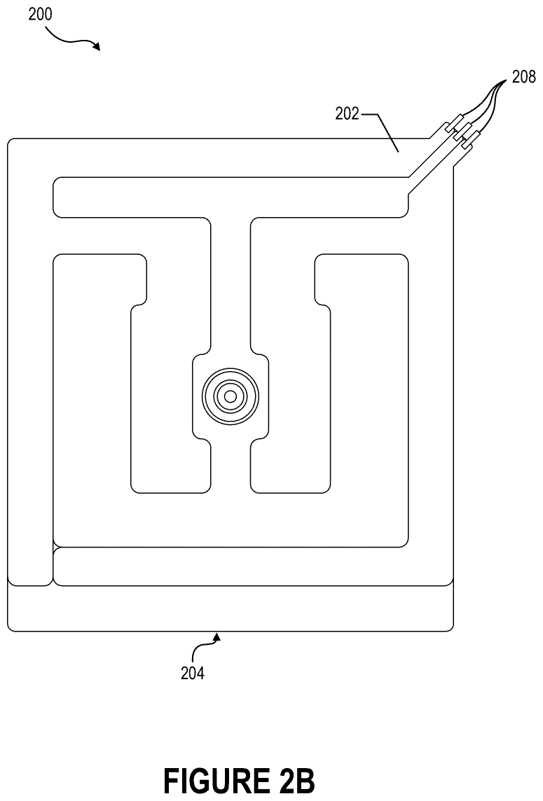

FIGS. 2A-2B are top and bottom views, respectively, of a pressure-mitigation device 200 configured in accordance with embodiments of the present technology. The pressure-mitigation device 200 is generally used in conjunction with non-elongated objects that support individuals in a seated or partially erect position. Examples of non-elongated objects include chairs (e.g., office chairs, examination chairs, recliners, and wheelchairs) and the seats included in vehicles and airplanes. Accordingly, the pressure-mitigation device 200 may be positioned atop surfaces that have side supports integrated into the object itself (e.g., the side arms of a recliner or wheelchair). Note, however, that the pressure-mitigation device 200 could likewise be used in conjunction with elongated objects in a manner generally similar to the pressure-mitigation device 100 of FIGS. 1A-1B.

In some embodiments, the pressure-mitigation device 200 is secured to a surface using an attachment apparatus. In other embodiments, the attachment apparatus is omitted such that the pressure-mitigation device 200 directly contacts the underlying surface. In such embodiments, the pressure-mitigation device 200 may have a tacky substance deposited along at least a portion of its outer surface that allows it to temporarily adhere to the surface.

The pressure-mitigation device 200 can include various features similar to the features of the pressure-mitigation device 100 described above with respect to FIGS. 1A-1B. For example, the pressure-mitigation device 200 may include a first portion 202 (also referred to as a “first layer” or “bottom layer”) designed to face the surface, a second portion 204 (also referred to as a “second layer” or “top layer”) designed to face the human body supported by the surface, and a plurality of chambers 206 formed via interconnections between the first and second portions 202, 204. In this embodiment, the pressure-mitigation device 200 includes an “M-shaped” chamber intertwined with a backward “J-shaped” chamber and a backward “C-shaped” chamber. Varying the pressure in such an arrangement of chambers 206 has been shown to effectively mitigate the pressure applied by a surface to the gluteal and sacral regions of a human body in a seated position. These chambers may be intertwined to collectively form a square-shaped pattern. Pressure-mitigation devices designed for deployment on the surfaces of non-elongated objects may have substantially quadrilateral-shaped patterns of chambers, while pressure-mitigation devices designed for deployment on the surfaces of elongated objects may have substantially square-shaped patterns of chambers.

As further discussed below, the chambers 206 can be inflated and/or deflated in a predetermined pattern and to predetermined pressure levels. The individual chambers 206 may be inflated to higher pressure levels than the chambers 106 of the pressure-mitigation device 100 described with respect to FIGS. 1A-B because the human body being supported by the pressure-mitigation device 200 is in a seated position, thereby causing more pressure to be applied by the underlying surface than if the human body were in a supine or prone position. Further, unlike the pressure-mitigation device 100 of FIGS. 1A-1B, the pressure-mitigation device 200 of FIGS. 2A-2B does not include side supports. As noted above, side supports may be omitted when the object on which the individual is situated (e.g., seated or reclined) already provides components that will laterally center the human body, as is often the case with non-elongated support surfaces. One example of such a component is the armrests along the side of a chair.

As further described below (e.g., with respect to FIGS. 11A-11C), a controller can control the pressure in each chamber 206 by providing a discrete airflow via one or more corresponding valves 208. Here, the pressure-mitigation device 200 includes three valves 208, and each of the three valves 208 corresponds to a single chamber 206. Other embodiments of the pressure-mitigation device 200 may include fewer than three valves or more than three valves, and each valve can be associated with one or more chambers to control inflation/deflation of those chamber(s). A single valve could be in fluid communication with two or more chambers. Further, a single chamber could be in fluid communication with two or more valves (e.g., one valve for inflation and another valve for deflation).

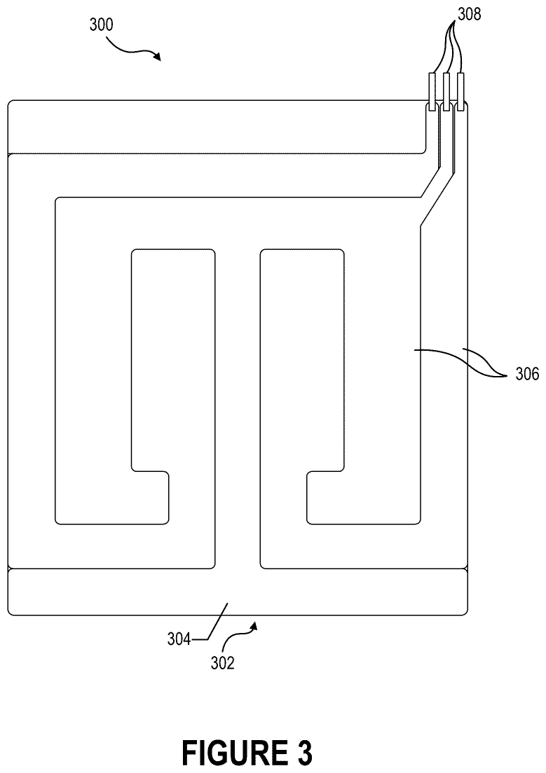

FIG. 3 is a top view of a pressure-mitigation device 300 for relieving pressure on an anatomical region applied by a wheelchair in accordance with embodiments of the present technology. The pressure-mitigation device 300 can include features similar to the features of the pressure-mitigation device 200 of FIGS. 2A-B and the pressure-mitigation device 100 of FIGS. 1A-1B described above. For example, the pressure-mitigation device 300 can include a first portion 302 (also referred to as a “first layer” or “bottom layer”) designed to face the seat of the wheelchair, a second portion 304 (also referred to as a “second layer” or “top layer”) designed to face the human body supported by the seat of the wheelchair, a series of chambers 306 formed by interconnections between the first and second portions 302, 304, and multiple valves 308 that control the flow of fluid into and/or out of the chambers 306. As can be seen in FIG. 3, the chambers 306 may be arranged similar to those shown in FIGS. 2A-2B. Here, however, the pressure-mitigation device 300 is designed such that the valves 308 will be located near the backrest of the wheelchair. Such a design may allow the tubing connected to the valves 308 to be routed through a gap near, beneath, or in the backrest.

In some embodiments the first portion 302 is directly adjacent to the seat of the wheelchair, while in other embodiments the first portion 302 is directly adjacent to an attachment apparatus. As shown in FIG. 3, the pressure-mitigation device 300 may include an “M-shaped” chamber intertwined with a “U-shaped” chamber and a “C-shaped” chamber, which are inflated and deflated in accordance with a predetermined pattern to mitigate the pressure applied to the sacral region of a human body in a sitting position on the seat of a wheelchair. These chambers may be intertwined to collectively form a square-shaped pattern.

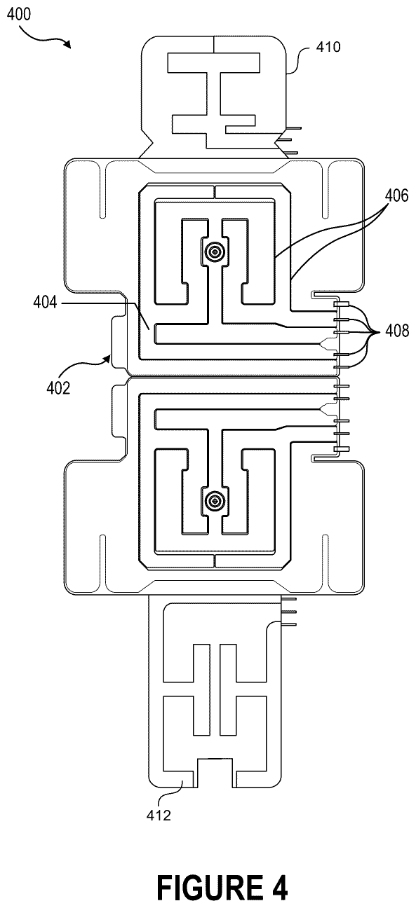

FIG. 4 is a top view of a pressure-mitigation device 400 for relieving pressure on an anatomical region applied by an elongated object in accordance with embodiments of the present technology. As mentioned above, examples of elongated objects include mattresses, stretchers, operating tables, and procedure tables. The pressure-mitigation device 400 can include features similar to the features of the pressure-mitigation device 300 of FIG. 3, the pressure-mitigation device 200 of FIGS. 2A-B, and the pressure-mitigation device 100 of FIGS. 1A-1B. For example, the pressure-mitigation device 400 can include a first portion 402 (also referred to as a “first layer” or “bottom layer”) designed to face the surface of the elongated object, a second portion 404 (also referred to as a “second layer” or “top layer”) designed to face a human body supported by the elongated object, a series of chambers 406 formed by interconnections between the first and second portions 402, 404, and multiple valves 408 that control the flow of fluid into and/or out of the chambers 406. As can be seen in FIG. 4, the pressure-mitigation device 400 may be designed such that the valves 408 will be accessible along a longitudinal side of the elongated object. Such a design may allow the tubing connected to the valves 408 to be routed alongside the elongated object (e.g., along or through a handrail of a bedframe). Alternatively, the pressure-mitigation device may be designed such that the valves 408 are located near the top or bottom of the pressure-mitigation device 400 so as to allow the tubing to be routed along a latitudinal side of the elongated object.

While some example pressure-mitigation devices described herein are designed to occupy the lumbar, gluteal, and femoral regions while the human body positioned thereon is in the supine position, the pressure-mitigation device 400 of FIG. 4 can be designed to also occupy cervical, thoracic, and leg regions. Thus, the pressure-mitigation device 400 may be able to alleviate pressure applied by the elongated object anywhere along the posterior side of the human body between the skull and ankle.

Embodiments of the pressure-mitigation device 400 could also include (i) a cranial portion 410 (also referred to as a “cranial cushion” or “cranial cup”) that is designed to envelop the posterior side of the cranium while the human body is in the supine position and/or (ii) a heel portion 412 (also referred to as a “heel cushion” or “heel cup”) that is designed to envelop the posterior end of the foot while the human body is in the supine position. The cranial portion 410 and heel portion 412 may include a different number of chambers than the geometric arrangements designed to occupy the lumbar and femoral regions. Generally, the cranial portion 410 and heel portion 412 only include one or two chambers, though the cranial portion 410 and heel portion 412 could include more than two chambers. In embodiments where the pressure-mitigation device 400 includes cranial and heel portions, the pressure-mitigation device 400 may be referred to as a “full-body pressure-mitigation device.” In embodiments where the pressure-mitigation device 400 includes cranial and heel portions, the pressure-mitigation device 400 may have a longitudinal form that is at least six feet in length. In embodiments where the pressure-mitigation device 400 does not include cranial and heel portions, the pressure-mitigation device 400 may have a longitudinal form that is at least four feet in length.

As shown in FIG. 4, the pressure-mitigation device 400 can include side supports 414 that are able to actively or passively orient the human body with respect to the chambers of the pressure-mitigation device 400. In some embodiments, a single side support extends longitudinally along each opposing side of the pressure-mitigation device 400. In other embodiments, multiple side supports are located along each opposing side of the pressure-mitigation device 400. As an example, along each longitudinal side, the pressure-mitigation device 400 may include a first side support that is intended to be parallel to the thoracic region and a second side support that is intended to be parallel to the leg region. As another example, along each longitudinal side, the pressure-mitigation device 400 may include a first side support that is intended to be parallel to the thoracic and lumbar regions, a second side support that is intended to be parallel to the leg region, and a third side support that is intended to be parallel to the calf region. Accordingly, the pressure-mitigation device 400 may include more than one side support along each side, and each side support may be responsible for orienting a different anatomical region of the human body.

More generally, the pressure-mitigation device 400 includes a first geometric arrangement of a first series of chambers and a second geometric arrangement of a second series of chambers. When controllably inflated, the first series of chambers can relieve the pressure applied to a first anatomical region of a human body by an underlying surface. Similarly, when controllably inflated, the second series of chambers can relieve the pressure applied to a second anatomical region of the human body by the underlying surface. When the pressure-mitigation device 400 has a longitudinal form as shown in FIG. 4, the first geometric arrangement can be longitudinally adjacent to the second geometric arrangement, so as to accommodate the first anatomical region that is superior to the second anatomical region. As shown in FIG. 4, the second geometric arrangement may be representative of another instance of the first geometric arrangement that is mirrored across a latitudinal axis that is orthogonal to the longitudinal form of the pressure-mitigation device 400. Alternatively, the second geometric arrangement may be identical to the first geometric arrangement.

Moreover, the pressure-mitigation device may include a third geometric arrangement of a third series of chambers. When controllably inflated, the third series of chambers can relieve the pressure applied to a third anatomical region of the human body by the underlying surface. The third anatomical region may be superior to the anatomical region (e.g., when the third geometric arrangement corresponds to the cranial portion 410), or the third anatomical region may be inferior to the second anatomical region (e.g., when the third geometric arrangement corresponds to the heel portion 412).

As mentioned above, the pressure-mitigation device could include cranial and heel portions in some embodiments. Therefore, the pressure-mitigation device may include a third geometric arrangement of a third series of chambers and a fourth geometric arrangement of a fourth series of chambers. When controllably inflated, the third series of chambers can relieve the pressure applied to a third anatomical region of the human body by the underlying surface. Similarly, when controllably inflated, the fourth series of chambers can relieve the pressure applied to a fourth anatomical region of the human body by the underlying surface. The third anatomical region may be superior to the first anatomical region, while the fourth anatomical region may be inferior to the second anatomical region.

### Overview of Approaches to Mitigating Pressure

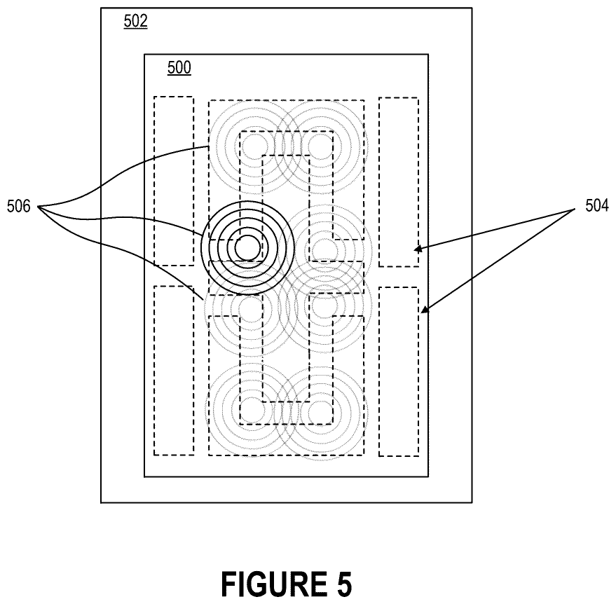

FIG. 5 is a partially schematic top view of a pressure-mitigation device illustrating how a pressure gradient can be created by varying pressure distributions to avoid ischemia in a mobility-impaired patient in accordance with embodiments of the present technology. When a human body is supported by a surface 502 of a substrate for an extended duration, pressure injuries may form in the tissue overlaying bony prominences, such as the skin overlying the sacrum, coccyx, heels, or hips. Generally, these bony prominences represent the locations at which the most pressure is applied by the surface 502 and, therefore, may be referred to as the “main pressure points” along the surface of the human body.

To prevent the formation of pressure injuries, healthy individuals periodically make minor positional adjustments (also known as “micro-adjustments”) to shift the location of the main pressure point. However, individuals having impaired mobility often cannot make these micro-adjustments by themselves. Mobility impairment may be due to physical injury (e.g., a traumatic injury or a progressive injury), movement limitations (e.g., within a vehicle, on an aircraft, or in restraints), medical procedures (e.g., those requiring anesthesia), and/or other conditions that limit natural movement. For these mobility-impaired individuals, the pressure-mitigation device 500 can be used to shift the location of the main pressure point(s) on their behalf. That is, the pressure-mitigation device 500 can create moving pressure gradients to avoid sustained, localized vascular compression and enhance tissue perfusion.

The pressure-mitigation device 500 can include a series of chambers 504 whose pressure can be individually varied. The chambers 504 may be formed by interconnections between the top and bottom layers of the pressure-mitigation device 500. The top layer may be comprised of a first material (e.g., a permeable, non-irritating material) configured for direct contact with a human body, while the bottom layer may be comprised of a second material (e.g., a non-permeable, gripping material) configured for direct contact with the surface 502. Generally, the first material is permeable to gasses (e.g., air) and/or liquids (e.g., water and sweat) to prevent buildup of fluids that may irritate the skin. Meanwhile, the second material may not be permeable to gasses or liquids to prevent soilage of the underlying object. Accordingly, air discharged into the chambers 504 may be able to slowly escape through the first material (e.g., naturally or via perforations) but not the second material, while liquids may be able to penetrate the first material (e.g., naturally or via perforations) but not the second material. Note, however, that the first material is generally be selected such that the top layer does not actually become saturated with liquid to reduce the likelihood of irritation. Instead, the top layer may allow liquid to pass therethrough into the cavities, from which the liquid can be subsequently discharged (e.g., as part of a cleaning process). The top layer and/or the bottom layer can be comprised of more than one material, such as a coated fabric or a stack of interconnected materials.

The pressure-mitigation device 500 may be designed such that inflation of at least some of the chambers 504 causes air to be continuously exchanged across the surface of the human body. Said another way, simultaneous inflation of at least some of the chambers 504 may provide a desiccating effect to inhibit generation and/or collection of moisture along the skin in a given anatomical region. In some embodiments, the pressure-mitigation device 500 is able to maintain airflow through the use of a porous material. For example, the top layer may be comprised of a biocompatible material through which air can flow (e.g., naturally or via perforations). In other embodiments, the pressure-mitigation device 500 is able to maintain airflow without the use of a porous material. For example, airflows can be created and/or permitted simply through varied pressurization of the chambers 504. This represents a new approach to microclimate management that is enabled by simultaneous inflation and deflation of the chambers 504. At a high level, each void formed beneath a human body due to deflation of at least one chamber can be thought of as a microclimate that cools and desiccates the corresponding portion of the anatomical region. Heat and humidity can lead to injury (e.g., further development of ulcers), so the cooling and desiccating effects may present some injuries due to inhibition of moisture generation/collection along the skin in the anatomical region.

In some embodiments, a pump (also referred to as a “pressure device”) can be fluidically coupled to each chamber 504 (e.g., via a corresponding valve) of each pressure-mitigation device, while a controller can control the flow of fluid generated by the pump into each chamber 504 on an individual basis in accordance with a predetermined pattern. The controller can operate the series of chambers 504 in several different ways.

In some embodiments, the chambers 504 have a naturally deflated state, and the controller causes the pump to inflate at least one of the chambers 504 to shift the main pressure point along the anatomy of the human body. For example, the pump may inflate at least one chamber located directly beneath an anatomical region to momentarily apply contact pressure to that anatomical region and relieve contact pressure on the surrounding anatomical regions adjacent to the deflated chamber(s). Alternatively, the controller may cause the pump to inflate two or more chambers adjacent to an anatomical region to create a void beneath the anatomical region to shift the main pressure point at least momentarily away from the anatomical region.

In other embodiments, the chambers 504 have a naturally inflated state, and the controller may cause deflation of at least one of the chambers 504 to shift the main pressure point along the anatomy of the human body. For example, the pump may cause deflation of at least one chamber located directly beneath an anatomical region, thereby forming a void beneath the anatomical region to momentarily relieve the contact pressure on the anatomical region. To deflate a chamber, the controller may simply prevent an airflow generated by the pump from entering the chamber as further discussed below. Additionally or alternatively, the controller may cause air contained in the chamber to be released (e.g., via a valve). At least partial deflation may naturally occur in this scenario if air escapes through the valve quicker than air enters the chamber.

Whether configured in a naturally deflated state or a naturally inflated state, the continuous or intermittent alteration of the inflation levels of the individual chambers 504 moves the location of the main pressure point across different portions of the human body. As shown in FIG. 5, for example, inflating and/or deflating the chambers 504 creates temporary contact regions 506 that move across the pressure-mitigation device 500 in a predetermined pattern, and thereby changing the location of the main pressure point(s) on the human body for finite intervals of time. Thus, the pressure-mitigation device 500 can simulate the micro-adjustments made by healthy individuals to relieve stagnant pressure applied by the surface 502.

The series of chambers 504 may be arranged in an anatomy-specific pattern so that when the pressure of one or more chambers is altered, the contact pressure on a specific anatomical region of the human body is relieved (e.g., by shifting the main pressure point elsewhere). As an example, the main pressure point may be moved between eight different locations corresponding to the eight temporary contact regions 506 as shown in FIG. 5. In some embodiments the main pressure point shifts between these locations in a predictable manner (e.g., in a clockwise or counter-clockwise pattern), while in other embodiments the main pressure point shifts between these locations in an unpredictable manner (e.g., in accordance with a random pattern or a semi-random pattern, based on the amount of force applied by the human body to the chambers, or based on the pressure of the chambers). Those skilled in the art will recognize that the number and position of these temporary contact regions 506 may vary based on the size of the pressure-mitigation device 500, the arrangement of chambers 504, the number of chambers 504, the anatomical region supported by the pressure-mitigation device 500, the characteristics of the human body supported by the pressure-mitigation device 500, the condition of the human body (e.g., whether the person is completely immobilized, partially immobilized, etc.), or any combination thereof.

As discussed above, the pressure-mitigation device 500 may not include side supports if the condition of a user (also referred to as the “patient” or “subject”) would not benefit from the positioning assistance provided by the side supports. For example, side supports can be omitted when the user is medically immobilized (e.g., under anesthesia, in a medically induced coma, etc.) and/or physically restrained on the underlying surface 502 (e.g., by rails on the side of a bed, arm rests on the side of a chair, restraints that limit movement, etc.).

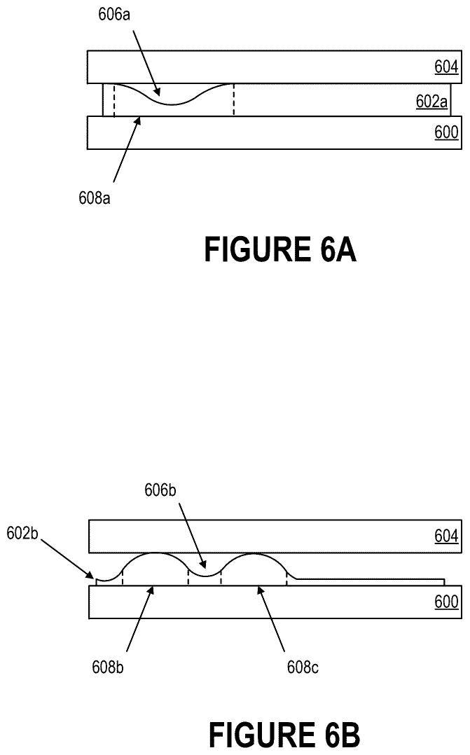

FIG. 6A is a partially schematic side view of a pressure-mitigation device 602a, for relieving pressure on a specific anatomical region by deflating one or more chambers in accordance with embodiments of the present technology. The pressure-mitigation device 602a can be positioned between the surface of an object 600 and a human body 604. Examples of objects 600 include elongated objects, such as mattresses, stretchers, operating tables, and procedure tables, and non-elongated objects, such as chairs (e.g., office chairs, examination chairs, recliners, and wheelchairs) and the seats included in vehicles and airplanes. To relieve the pressure on a specific anatomical region of the human body 604, at least one chamber 608a of multiple chambers (collectively referred to as “chambers 608”) proximate to the specific anatomical region is at least partially deflated to create a void 606a beneath the specific anatomical region. In such embodiments, the remaining chambers 608 may remain inflated. Thus, the pressure-mitigation device 602a may sequentially deflate chambers (or arrangements of multiple chambers) to relieve the pressure applied to the human body 604 by the surface of the object 600.

FIG. 6B is a partially schematic side view of a pressure-mitigation device 602b for relieving pressure on a specific anatomical region by inflating one or more chambers in accordance with embodiments of the present technology. For example, to relieve the pressure on a specific anatomical region of the human body 604, the pressure-mitigation device 602b can inflate two chambers 608b and 608c disposed directly adjacent to the specific anatomical region to create a void 606b beneath the specific anatomical region. In such embodiments, the remaining chambers may remain partially or entirely deflated. Thus, the pressure-mitigation device 602b may sequentially inflate a chamber (or arrangements of multiple chambers) to relieve the pressure applied to the human body 604 by the surface of the object 600.

The pressure-mitigation devices 602a, 602b of FIGS. 6A-6B are shown to be in direct contact with the contact surface. However, in some embodiments, an attachment apparatus is positioned between the pressure-mitigation devices 602a, 602b and the object 600. The attachment apparatus may be designed to help secure the pressure-mitigation devices 602a, 602b and the object 600. For example, the attachment apparatus may be made of a material that is naturally tacky or sticky so as to inhibit movement of the pressure-mitigation devices 602a, 602b with respect to the object 600. Alternatively, the bottom side of the pressure-mitigation devices 602a, 602b could be coated with a material, such as a removable adhesive (e.g., an elastomer- or silicone-based sealant or a pressure-sensitive film) or tacky substance (e.g., silicone rubber).

In some embodiments, the pressure-mitigation devices 602a, 602b of FIGS. 6A-6B have the same configuration of chambers 608 and can operate in both a normally inflated state (described with respect to FIG. 6A) and a normally deflated state (described with respect to FIG. 6B) based on the selection of an operator (e.g., the user or some other person, such as a medical professional or family member). For example, the operator can use a controller to select a normally deflated mode such that the pressure-mitigation device operates as described with respect to FIG. 6B, and then change the mode of operation to a normally inflated mode such that the pressure-mitigation device operates as described with respect to FIG. 6A. Thus, the pressure-mitigation devices described herein can shift the location of the main pressure point by controllably inflating chambers, controllably deflating chambers, or a combination thereof.

While the embodiments disclosed above involve a separate pressure-mitigation device (e.g., pressure-mitigation devices 602a, 602b) being placed atop a contact surface of a substrate (e.g., object 600), embodiments disclosed below directly integrate the pressure-mitigation device with the substrate. By way of the following embodiments, an amount of equipment needed to perform the pressure-mitigation treatment can be reduced. The unitary pressure-mitigation substrate can then be a portable unit and can be transported to different locations. For example, a unitary pressure-mitigation pad can be used by a patient in many different environments (e.g., on bleachers at a sporting event and at home on a chair).

### Overview of Device Modularity

Embodiments of the disclosed technology introduce modularity and dynamic usage of pressure-mitigation devices for different body sizes and in different environments. Modular components of a pressure-mitigation device can be arranged, added, removed, and/or the like to fit a pressure-mitigation device for a particular patient and/or for a particular environment. For example, a pressure-mitigation device is combined with additional modular components and/or other pressure-mitigation devices to form an aggregate pressure-mitigation device with a larger surface area across which pressure experienced by a human body thereon can be alleviated. As another example, modular components of a pressure-mitigation device are removed from the pressure-mitigation device to reduce a surface area or size of the pressure-mitigation device to adapt the pressure-mitigation device for a smaller body (e.g., a neonatal or pediatric body) and/or for a smaller environment (e.g., a chair). The modular components can be arranged adjacently with one another to form a continuous surface having a geometric form and arrangement of chambers via which a pressure gradient can be applied on a body disposed atop the continuous surface. Various embodiments of modular pressure-mitigation devices are suitable and adaptable between full-body applications, partial-body applications, neonatal applications, adult applications, and the like. In some embodiments, a pressure-mitigation device is constructed from multiple modular components to have a size suited for a particular substrate, such as a mattress, a chair seat, a wheelchair, and/or the like. As such, pressure-mitigation treatment can be adaptably provided in different settings based on dynamically arranging pressure-mitigation devices.

According to some embodiments, modularity of pressure-mitigation devices is realized via separate and individual modular chamber devices that operate independently. Each modular chamber device includes an inflatable chamber and is configured to inflate or deflate the inflatable chamber, in concert with other modular chamber devices, to provide a pressure-mitigation treatment for a body disposed atop the modular chamber device and the other modular chamber devices. Some modular chamber devices include more than one chamber, such as two inflatable chambers, three inflatable chambers, four inflatable chambers, and/or the like. In some embodiments, a modular chamber device includes a fewer number of inflatable chambers than the pressure-mitigation devices discussed above (e.g., the pressure-mitigation devices of FIGS. 1A-4). Each modular chamber device may include a respective controller, a respective pump and fluid egress/ingress, and other components disclosed herein with respect to chamber inflation and deflation. Thus, a controller of a modular chamber device is responsible for one inflatable chamber, or more than one inflatable chamber depending on the configuration of the modular chamber device. Multiple modular devices can be connected together in a geometric arrangement to form an aggregate pressure-mitigation device, resembling the pressure-mitigation devices discussed above (e.g., the pressure-mitigation devices of FIGS. 1A-4). When arranged and connected together, a particular controller can become responsible for controlling its respective modular chamber device as well as other modular chamber devices. For example, in the aggregate pressure-mitigation device, one of the multiple controllers becomes a central controller that provides commands or instructions to the other multiple controllers. In another example, an aggregate pressure-mitigation device includes multiple lead controllers each corresponding to and responsible for a region or portion of the aggregate pressure-mitigation device.

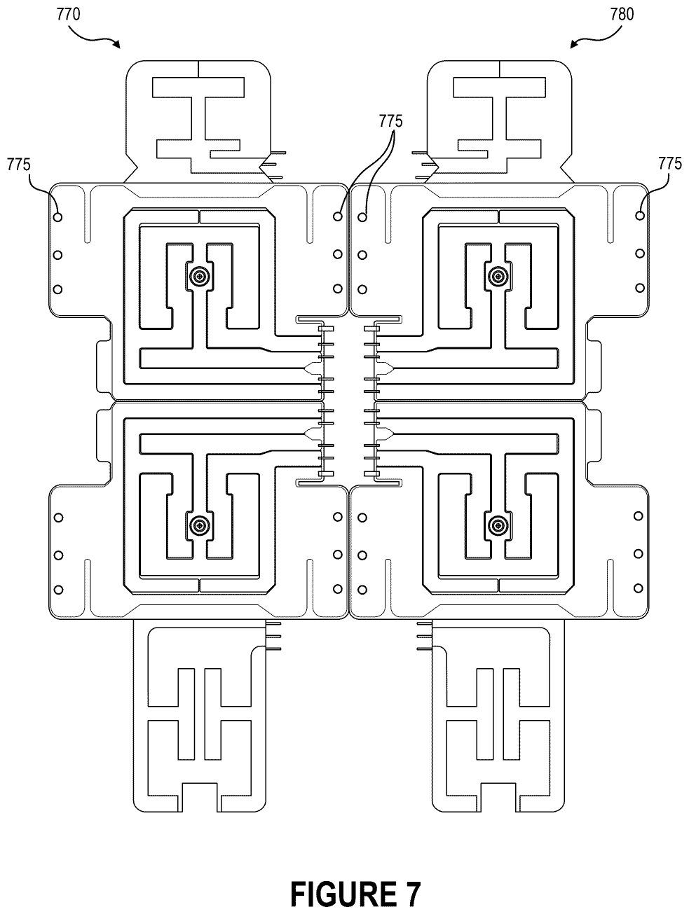

FIG. 7 illustrates an example embodiment of modular adaptability with pressure-mitigation devices. In particular, FIG. 7 shows how multiple pressure-mitigation devices 770, 780 can be connected to one another. Each type of pressure-mitigation device described herein may be designed to be detachably connectable to the same type of pressure-mitigation device and/or a different type of pressure-mitigation device. For example, a pressure-mitigation device designed for non-elongated objects could be detachably connected alongside another pressure-mitigation device designed for non-elongated objects, or a pressure-mitigation device designed for non-elongated objects could be detachably connected alongside a pressure-mitigation device designed for elongated objects. Similarly, a pressure-mitigation device designed for elongated objects could be detachably connected alongside another pressure-mitigation device designed for elongated objects. Thus, multiple human bodies (e.g., related persons, such as a husband and wife) could be deployed alongside one another (e.g., in a single bed, in adjacent seats of a vehicle, etc.).

Pressure-mitigation devices can be detachably connected to one another using different forms of attachment mechanisms 775. As an example, a pressure-mitigation device may have a longitudinal form that is defined by opposing longitudinal sides, and the pressure-mitigation device may include at least one attachment mechanism along a first longitudinal side of the opposing longitudinal sides and at least one attachment mechanism along a second longitudinal side of the opposing longitudinal sides. The attachment mechanisms could be magnets, where the magnets arranged along the first longitudinal side have opposite polarity of the magnets arranged along the second longitudinal side. Specifically, magnets of one pole (e.g., north) may be located along one longitudinal side, while magnets of the other pole (e.g., south) may be located along the other longitudinal side. When pressure-mitigation devices are placed in proximity to one another, the magnets may naturally be attracted to one another. As another example, a pressure-mitigation device may include one or more mechanical structures, such as zippers, buttons, clasps, and the like, arranged along each longitudinal side. As another example, a pressure-mitigation device may include an adhesive film arranged along each longitudinal side. As another example, a pressure-mitigation device may include strips of hook-and-loop fasteners (e.g., made by VELCRO®) along each longitudinal side.

Assume that a pair of pressure-mitigation devices are to be secured to one another. In some embodiments, the pair of pressure-mitigation devices operate independently despite being detachably connected to one another. Thus, each pressure-mitigation device may be connected to its own controller. In other embodiments, the pair of pressure-mitigation devices operate together as a single unit. Thus, the pair of pressure-mitigation devices may be connected to a single controller that is responsible for controlling fluid flow into the chambers of each pressure-mitigation device. For example, multi-channel tubing that is connected to the controller may split along one end, and one split end may be fluidically coupled to a first pressure-mitigation device while another split end may be fluidically coupled to a second pressure-mitigation device. Such an approach allows the controller to simultaneously control the first and second pressure-mitigation devices.

Thus, some embodiments provide modularity based on connection of multiple pressure-mitigation devices that each include a plurality of inflatable chambers and are independently and individually operable. Embodiments of the disclosed technology further provide chamber-wise modularity and chamber independence for a given pressure-mitigation device. In particular, chamber-wise modularity and adaptability of a pressure-mitigation device provides increased spatial resolution when customizing the geometric arrangement of inflatable chambers of the pressure-mitigation device. For example, an ability to add or remove individual inflatable chambers from a pressure-mitigation device enables the pressure-mitigation device to fit a body and/or an environment more precisely than connecting the pressure-mitigation device to other entire pressure-mitigation devices.

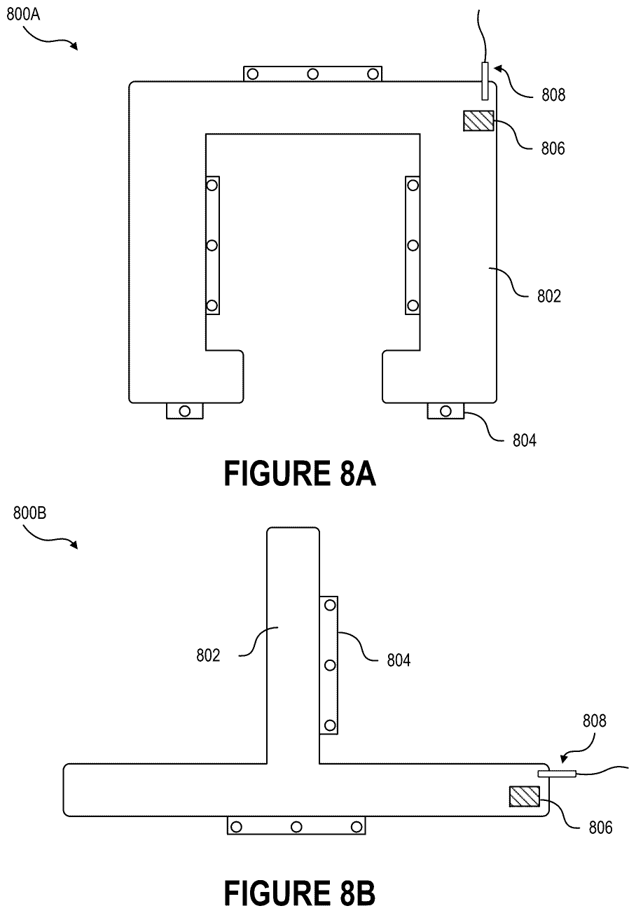

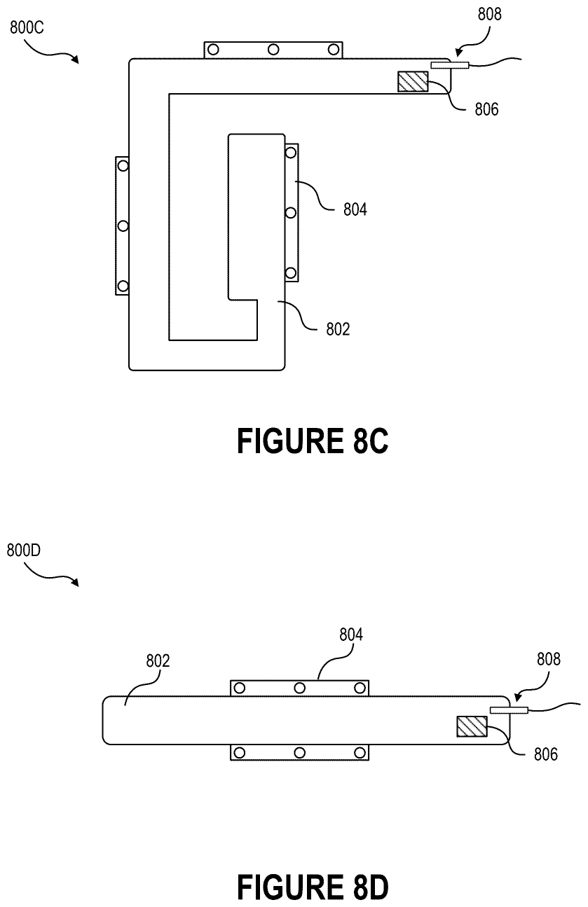

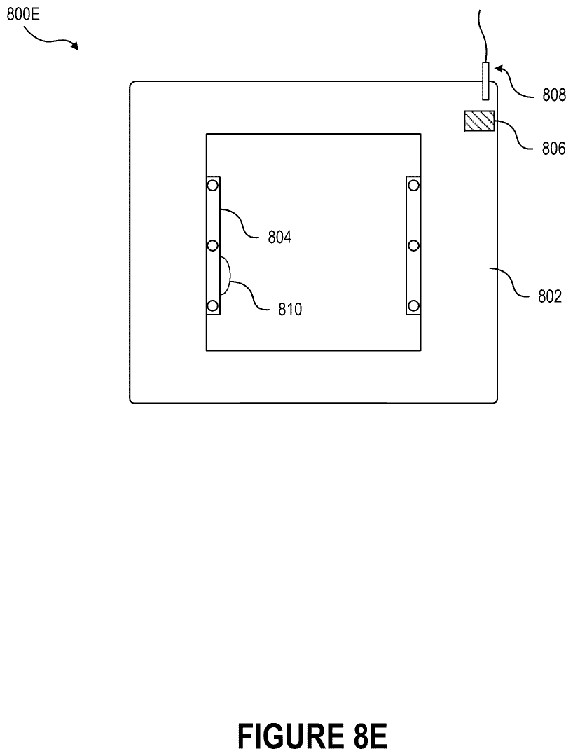

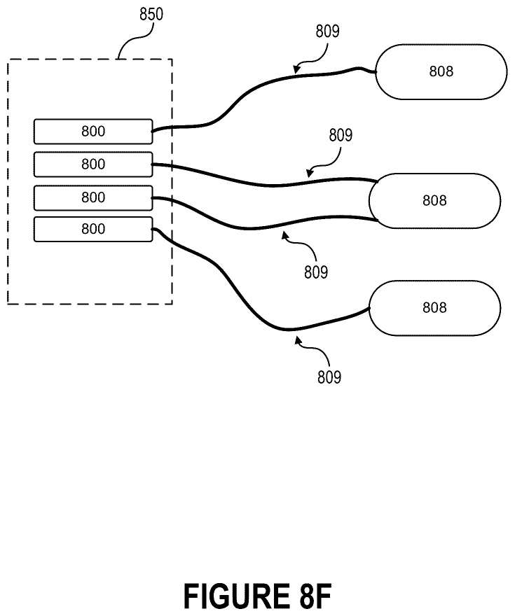

FIGS. 8A-8E illustrate example modular chamber devices that may be arranged and connected together to form an aggregate pressure-mitigation device. As shown, each of the modular chamber devices 800 illustrated across FIGS. 8A-8E are configured with a particular sectional shape. For example, a first modular chamber device 800A is configured with a “C” shape, a second modular chamber device 800B is configured with a “T” shape, a third modular chamber device 800C is configured with a “J” shape, a fourth modular chamber device 800D is configured with an “I” shape, and a fifth modular chamber device 800E is configured with an “O” shape. It will be understood that, beyond these five illustrated examples, other modular chamber devices 800 may be configured with other sectional shapes (e.g., an “M” shape, a backwards “J” shape, a “U” shape, and other shapes that may be described herein).

In particular, a sectional shape of a modular chamber device 800 refers to a geometry of the inflatable chamber 802 of the modular chamber device 800, for example from a top or bottom perspective. The sectional shape of a modular chamber device 800 may be persistent such that the inflatable chamber 802 retains the sectional shape during and after inflation and deflation. Similar to other embodiments disclosed herein, the inflatable chamber 802 of a modular chamber device 800 may be formed by interconnections between an upper layer and a lower layer. In some embodiments, the inflatable chamber 802 of a modular chamber device 800 is formed of a single construction. In some embodiments, the modular chamber device 800 includes a single inflatable chamber. In some embodiments, a modular chamber device 800 includes one inflatable chamber, two inflatable chambers, three inflatable chambers, four inflatable chambers, and/or the like.

In some embodiments, a sectional shape of a modular chamber device 800 is configured to fit with more of the same sectional shape and/or with other sectional shapes. For example, the “I” shape of the fourth modular chamber device 800D is configured to fit flush next to other modular chamber devices 800 also having the “I” shape, as well as fitting flush next to a distal side of an elongated portion of a modular chamber device 800 having the “C” shape. Various fittings between different shapes are extensive and can vary in complexity. In the example of multiple “I” shape modular chamber devices fitting next to one another, each modular chamber device fits next to another along one edge of the “I” shape. In other examples, a portion of the “C” shape of the first modular chamber device 800A is configured to fit within a partially enclosed area of the “J” shape of the third modular chamber device 800C, and in doing so, multiple external edges of the “C” shape are flush against multiple internal edges of the “J” shape.

As an illustrative example, an “M” shaped modular chamber device, a backwards “J” shaped modular chamber device, and a backwards “C” shaped modular chamber device are arranged and intertwined to form an aggregate pressure-mitigation device with the same geometric arrangement of inflatable chambers as the pressure-mitigation device 200 of FIGS. 2A-2B. As another illustrative example, a plurality of “I” shaped modular chamber devices are arranged and aligned lengthwise adjacent to one another to form an aggregate pressure-mitigation device that can be operated to carry out a pressure “wave” that can be applied on a body (e.g., a calf or leg region of a human body).

As discussed, the sectional shapes of modular chamber devices 800 can be fitted with those of other modular chamber devices 800 to be arranged into a geometric arrangement of inflatable chambers, and thereby forming an aggregate pressure-mitigation device. As a result, the geometric arrangement of chambers in an aggregate pressure-mitigation device may resemble those of the pressure-mitigation devices discussed and illustrated previously, for example at FIGS. 1A-3. For example, an aggregate pressure-mitigation device that geometrically resembles the pressure-mitigation device 200 of FIG. 2A may be formed by arranging one or more “C” shape modular chamber devices, one or more “J”-shape modular chamber devices, one or more “I”-shape modular chamber devices, an “M”-shape modular chamber device, and/or the like together. And because an aggregate pressure-mitigation device is dynamically formed based on the arrangement of multiple modular chamber devices, additional modular chamber devices can be further added to the aggregate pressure-mitigation device to increase a spatial span of the aggregate pressure-mitigation device, and some of the already-arranged modular chamber devices can be removed to decrease the spatial span of the aggregate pressure-mitigation device.

For example, the “O” shape of the fifth modular chamber device 800E is configured as an omni-directional spacer for generally increasing a span of an aggregate pressure-mitigation device in multiple directions. For example, the fifth modular chamber device 800E may be fitted around an arrangement of other modular chamber devices to increase the span of the arrangement in multiple directions. In some embodiments, multiple variations or levels of the fifth modular chamber device 800E with different internal and outer diameters may be used to hierarchically increase or decrease an omni-directional span of an arrangement of modular chamber devices. The resulting arrangement of multiple “O” shaped modular chamber devices with different diameters may then resemble nested rings and allows for convenient and rapid management of the spanned area of an aggregate pressure-mitigation device. Functionally, controlled inflation of nested “O” shaped modular chamber devices can assist in the positioning of a body over the aggregate pressure-mitigation device. In an example, an outermost “O” shaped modular chamber device is maximally inflated, and each inner “O” shaped modular chamber device is less inflated, resulting in a cup or crater that assists in positioning a body over a center of the aggregate pressure-mitigation device.

In some embodiments, the aggregate pressure-mitigation device is formed by positioning and placing multiple modular chamber devices adjacent to one another in a geometric arrangement, and by virtue of a body being placed atop the aggregate pressure-mitigation device, the multiple modular chamber devices may be physically limited in separating from the geometric arrangement. In some embodiments, the modular chamber devices 800 composing an aggregate pressure-mitigation device are physically interconnected via attachment mechanisms 804. The attachment mechanisms 804 may located along one or more edges of the sectional shape of a modular chamber device 800 and may be configured to interface with and connect to corresponding attachment mechanisms 804, for example those located on other modular chamber devices 800, and/or other surfaces, for example those located on other modular chamber devices 800.

According to some embodiments, attachment mechanisms 804 for a modular chamber device 800 include various attachment mechanisms disclosed herein (e.g., attachment mechanisms 775). For example, the attachment mechanisms 804 that facilitate interconnection between modular chamber devices include magnets, mechanical structures (e.g., zippers, buttons, clasps, straps, and the like), adhesive films, tacky substances and/or surfaces, hook-and-loop fasteners, and/or the like.

In some embodiments, each edge of the sectional shape of a modular chamber device 800 includes an attachment mechanism 804, and a modular chamber device 800 may include different types of attachment mechanisms 804 depending on edge locations. For example, with respect to the “C” shape of the first modular chamber device 800A, a first type of attachment mechanism 804 that is associated with a high detachment threshold (e.g., a button, a zipper, magnets with high strength) is located on externally-facing edges of the “C” shape, while a second type of attachment mechanism 804 that is associated with a relatively lower detachment threshold (e.g., adhesive films, hook-and-loop fasteners, magnets with lower strength) is located on internally-facing edges of the “C” shape. Because internally-facing edges of the “C” shape physically confine or enclose at least partially other modular chamber devices 800 fitted within, attachment mechanisms with lower detachment thresholds may be sufficient and may allow for conservation of materials and resources when constructing a modular chamber device 800. In some embodiments, attachment mechanisms 804 are located on the externally-facing edges of modular chamber devices 800 configured with concave sectional shapes, and the internally-facing edges thereof do not include attachment mechanisms 804.

Indeed, certain attachment mechanisms may be associated with readily detachable modular chamber devices, while other attachment mechanisms may be associated with fixedly attachable modular chamber devices. In some examples, modular chamber devices configured with attachment mechanisms 804 for detachability (e.g., magnets, zippers) are used in place of modular chamber devices configured with attachment mechanisms 804 for fixed attachment (e.g., adhesive films) in applications and settings where the body size of bodies receiving pressure-mitigation treatment vary more. For example, neonatal and/or pediatric bodies can vary in size, and thus, modular chamber devices for use in neonatal and/or pediatric applications can include attachment mechanisms 804 for detachability for easier arrangement and adaptability between treatment subjects.

In addition to, or alternative to, the attachment mechanisms 804, the modular chamber devices 800 may be arranged atop a backing that assists in maintaining and securing a geometric arrangement of the modular chamber devices 800. For example, the backing includes a tacky or sticky surface that resists movement and sliding of the modular chamber devices 800, thereby securing the modular chamber devices 800 in their arrangement. For example, the backing is a silicone or rubber mat. Similarly, each modular chamber device 800 may be constructed to include adhesive or tacky surfaces such that a modular chamber device 800 is able to sufficiently adhere to an underlying surface on its own. In some embodiments, a mapped backing is used to arrange and secure modular chamber devices 800. The mapped backing may be a mat (e.g., with or without a tacky or sticky surface) that includes indications that assist a user to arrange the modular chamber device in a predetermined arrangement. For example, the mat may include printed or drawn outlines for specific shapes of modular chamber devices, which guide a user to place specific modular chamber devices within the outlines. In some embodiments, the indications of the mapped backing are physically raised walls or ridges, such that the mapped backing includes shells in which the modular chamber devices are rested. Different mapped backings may be associated with different applications in which pressure-mitigation treatment is used and may serve as useful guidance tools for users to arrange and also secure modular chamber devices.

In addition to the ability to be arranged and/or connected with other modular chamber devices, a given modular chamber device may be configured to be independently and individually operable. In particular, the given modular chamber device may be configured for inflation and deflation of its respective inflatable chamber 802, regardless of whether the given modular chamber device is arranged and/or connected with other modular chamber devices. As such, a single modular chamber device may be used by itself to provide a pressure-mitigation treatment, or other comfort-related, mitigation-related, and/or stability-related function. For example, an “O”-shaped or a “C”-shaped modular chamber device may be used individually as a cranial cup that supports and elevates a head or skull region of a human body based on the inflation and/or deflation of the respective “O”-shaped or “C”-shaped inflatable chamber. As another example, an “I”-shaped modular chamber device can be used individually as a support for a lumbar region of a human body and/or as an elevating support for the ankles of the human body based on the inflation and/or deflation of the “I”-shaped inflatable chamber.

In accordance with the individual operation capability of modular chamber devices 800, a modular chamber device 800 may include a controller 806 and pump 808. Via the controller 806 and the pump 808, the inflatable chamber 802 can be inflated and/or deflated to operate the modular chamber device 800, whether individually or in concert with other modular chamber devices 800 arranged with the modular chamber device 800. Thus, with respect to an aggregate pressure-mitigation device, each modular chamber device 800 thereof can be individually operated by respective controllers and pumps 808.

FIG. 8F illustrates a diagram that shows an aggregate pressure-mitigation device 850 being formed from a plurality of modular chamber devices 800. According to aspects of the disclosed technology, the modularity of the aggregate pressure-mitigation device 850 is realized by way of a lack of intervening tubing between the modular chamber devices 800. In particular, the lack of intervening tubing between modular chamber devices 800 facilities the independence between modular chamber devices 800 and corresponds to the ability of a modular chamber device 800 to operate independently whether as part of an aggregate pressure-mitigation device 850 or otherwise. Thus, as shown in the illustrated example, each modular chamber device 800 can include its own respective tubing 809 that can connect to a respective pump device 808, fluid supply, controller/pump assembly, and/or the like. Due to the lack of intervening tubing, fluid connection failures and leaks can be quarantined or contained to a given modular chamber device and may not propagate to lead to a complete fault of the aggregate pressure-mitigation device 850. In some embodiments, a pump device 808 includes multiple ports that each are connectable with a tubing 809 of the aggregate pressure-mitigation device, and thus, a number of pump devices 808 needed to operate the aggregate pressure-mitigation device may be less than the number of modular chamber devices 800.

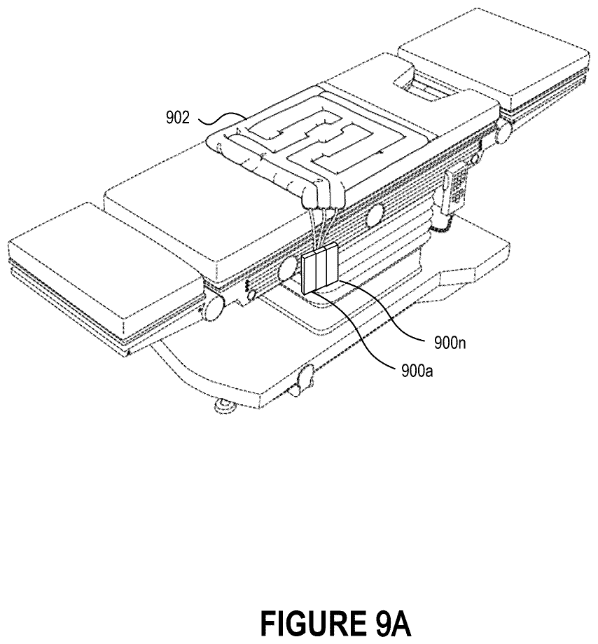

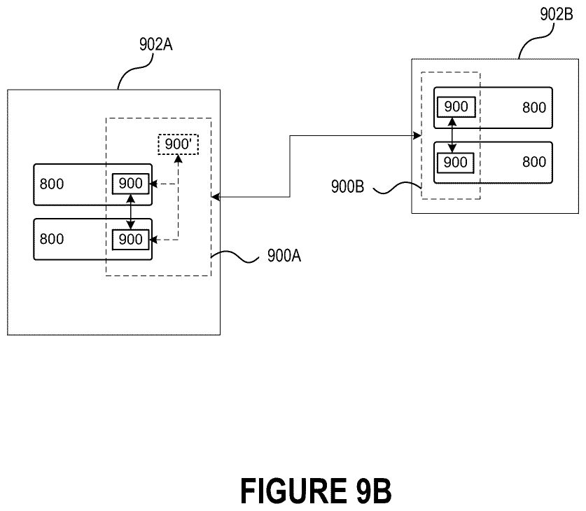

FIG. 9A illustrates how aspects of the controller and pump may be incorporated into modular assemblies 900a-n, for example, for a pressure-mitigation device or for an aggregate pressure-mitigation device. In such embodiments, the pump that supplies the flow of fluid that is manipulated to inflate the chambers of a pressure-mitigation device 902 can be part of the controller. As shown in FIG. 9A, these modular assemblies 900a-n can be detachably connected to the pressure-mitigation device 902 as necessary, and then removed when the pressure-mitigation device 902 is no longer being used.

In some embodiments, the number of modular assemblies needed to controllably inflate a given pressure-mitigation device is based on the number of channels into which fluid can flow. In FIG. 9A, for example, the pressure-mitigation device 902 includes three channels for the three chambers, as the pressure-mitigation device 902 does not include side supports. Each modular assembly can be designed to support a predetermined number of channels. For example, modular assemblies may be designed to support a single channel, or modular assemblies may be designed to support more than one channel (e.g., two or three channels). In some embodiments, the number of modular assemblies 900 corresponds to the number of modular chamber devices composing an aggregate pressure-mitigation device, with each modular chamber device including a modular controller-pump assembly.

In other embodiments, the number of modular assemblies needed to controllably inflate a given pressure-mitigation device is based on a characteristic of a human body to be situated thereon and/or a characteristic of the surface on which the given pressure-mitigation device is to be deployed. For example, each modular assembly may be “weight rated” for a certain number of pounds, and the number of modular assemblies that are needed may depend on the weight of the human body.

Note that, in some embodiments, these modular assemblies 900a-n can be attached directly to the pressure-mitigation device 902 without any intervening tubing. In such embodiments, each modular assembly may have one or more attachment mechanisms located around its egress fluid interface, and the pressure-mitigation device 902 may have one or more attachment mechanisms located around each of its ingress fluid interfaces. Normally, these ingress fluid interfaces are located in easily reachable places. For example, the ingress fluid interfaces may be located around the periphery of the pressure-mitigation device as shown in FIGS. 1A-4. Thus, the ingress fluid interfaces may be located in “flaps” or “extensions” that extend off the underlying surface on which the human body and pressure-mitigation device are situated. These “flaps” or “extensions” may extend the chambers outside of the geometrical pattern to be oriented beneath the human body.

As an example, assume that the pressure-mitigation device 902 has multiple ingress fluid interfaces through which fluid is able to flow into corresponding chambers. Each ingress fluid interface may have magnets arranged about its periphery. Each modular assembly may have a complementary arrangement of magnets about the periphery of its egress fluid interface. When a modular assembly is brought within proximity of a given ingress fluid interface of the pressure-mitigation device 902, the complementary arrangements of magnets can attract one another. Thus, the egress fluid interface of the modular assembly and the ingress fluid interface of the pressure-mitigation device 902 can be detachably connected to one another without intervening tubing. Other examples of attachment mechanisms include clips, clasps, buttons, latches, patches of hook-and-loop fasteners, adhesives, and the like. Note that while this feature is described in the context of modular assemblies, a non-modular controller (e.g., the controller 1100 of FIGS. 11A-11C) could also be attached directly to a pressure-mitigation device without any intervening tubing.

As discussed, individual modular chamber devices composing an aggregate pressure-mitigation device include respective controllers and/or modular assemblies 900, and the respective controllers and/or modular assemblies 900 may coordinate and communicate each other to operate multiple modular chamber devices in concert. In some embodiments, a controller is configured to communicate information local to the respective modular chamber device to controllers of other modular chamber devices, and in particular, to controllers of other modular chambers that are arranged and/or connected to the respective modular chamber device. In some embodiments, for example, a controller executes computer-readable code that causes the controller to transmit, to one or more other controllers, an indication of an inflation/deflation state of the respective modular chamber device, a measured pressure exerted by a body on the respective modular chamber device, an error or fault related to inflation/deflation operation of the respective modular chamber device, and/or the like.

In some embodiments, controllers communicate with each other via wireless communication. For example, a controller transmits information to, and receives information from, other controllers via Bluetooth communication, Bluetooth Low Energy (BLE) communication, a local area network (LAN) (e.g., a Wi-Fi network), a cellular telecommunication network (e.g., a 3G network, a 4G LTE network, a 5G NR network, a 6G network), an Internet of Things (IoT) network, radio frequency identification (RFID) communication, near-field communication (NFC), and/or other mediums, protocols, and networks not explicitly listed here. In some embodiments, the controllers of an aggregate pressure-mitigation device are coupled to one another via a wired connection, such that a controller may transmit information to, and receive information from, other controllers via wired communication. Accordingly, in some embodiments, each controller includes hardware sockets, ports, dongles, and/or the like that can be connected to various example cables and connectors, such as Universal Serial Bus (USB) cables.

In order for controllers to communicate with other controllers, a controller may register and/or identify other controllers that belong to modular chamber devices that are arranged and/or connected with a respective modular chamber device. With respect to controllers communicating over wired communication with one another, registration information can be passed between the controllers such that one or more controllers are able to identify each of the controllers belonging to the aggregate pressure-mitigation device. In some embodiments, with respect to wireless communication between controllers, a controller is configured to wireless broadcast a message (e.g., based on user input) that indicates arrangement and inclusion of a respective modular chamber device in an aggregate pressure-mitigation device. With each controller included in an aggregate pressure-mitigation device broadcasting its presence and inclusion in the aggregate pressure-mitigation device, the controllers can be aware of each other, and of a number of inflatable chambers included in the aggregate pressure-mitigation device.

In some embodiments, a controller of a modular chamber device is configured to determine a distance between the controller and another controller based on the receipt of wireless signals from the other controller. Accordingly, in such embodiments, controllers can identify other controllers belonging to the same aggregate pressure-mitigation device based on a determined distance between controllers. In particular, if a controller determines that another controller is within a predetermined threshold distance, then the controller may determine that the other controller belongs to the same aggregate pressure-mitigation device.

To facilitate coordinated or concerted operation of the inflatable chambers of the aggregate pressure-mitigation device, one or more of the controllers may transmit instructions to other controllers to cause various inflatable chambers to inflate or deflate in accordance with a pre-determined pattern. In some embodiments, a lead/master/central controller is designated among the controllers, and the lead controller is configured to transmit instructions to other controllers to control operation of their respective inflatable chambers. Instructions originating from the lead controller may be associated with a higher priority compared to instructions locally determined by a given controller, and lead controller instructions received by the given controller may override instructions that are locally determined by the given controller. Depending on the embodiment, various consensus protocols, voting protocols, and/or the like may be implemented by the multiple controllers of the aggregate pressure-mitigation device in order for a lead controller to be selected. In some embodiments, at least a subset of the controllers of an aggregate pressure-mitigation device are communicably coupled to a separate controller (e.g., a controller according to FIGS. 11A-11C), which acts as the lead/master/central controller that distributes and relays instructions or commands among the controllers belonging to the aggregate pressure-mitigation device.

In some embodiments, the controllers are configured to cooperate based on maintaining open and real-time communication with one another. For example, in response to a given controller carrying out a command to inflate or deflate its respective inflatable chamber(s), the given controller also reports the command to the other controllers, so that the other controllers are aware that the given controller is in the process of inflating or deflating its respective chamber(s). In some embodiments, a controller operates its respective inflatable chamber(s) according to a cycle or waveform, and the controller reports to the other controllers at certain predetermined timepoints in the cycle or waveform (e.g., at a beginning of the cycle, at an ending of the cycle, at a midpoint of the cycle). In some embodiments, these real-time status updates and other real-time communications are relayed by a given controller to each of the other controllers. In some embodiments, the given controller communicates its real-time status updates and other real-time information to a lead/master/central controller that manages communication between the multiple controllers. Via open and real-time communication, precise timing and coordination in the independent operation of multiple inflatable chambers is enabled. Additionally, errors, faults, cycle or timing deviations, and/or the like are more readily detected via the open and real-time communication and can be preemptively addressed. For example, based on real-time updates received from a first controller, a central controller can determine that the first controller is becoming out of sync with a second controller such that an intended pressure gradient would not be applied on a body via a respective first chamber and a respective second chamber. Based on the determination, the central controller can initiate a resynchronization process for the first controller, and/or command the first controller to adjust a timing or cycle.

Some other embodiments minimize continuous and excessive inter-controller communication, thereby lightening communication and processing load on each controller and/or a central controller. In some embodiments, individual controllers inflate/deflate respective inflatable chambers according to preset frequencies based on an initial synchronization. Indeed, each individual controller may be configured to inflate and deflate respective inflatable chambers according to a pattern, as per a modular chamber device being individually operable and usable. In some embodiments, the controllers perform an initial synchronization with one another, such that adjacent chambers that belong to different modular chamber devices are not concurrently inflated in a way that precludes the creation of a void therebetween (if such a void is needed at a particular instance of the pressure-mitigation treatment, as discussed in connection with FIGS. 6A-6B). For example, the controllers may communicate with one another to initiate respective inflation/deflation patterns that are orthogonal with neighboring patterns (e.g., with respect to time-wise inflation volume waveforms). Then, following the initial synchronization, the controllers may adhere to the respective inflation/deflation patterns, and inter-controller communication may be obviated to an extent. In some embodiments, the controllers are configured to resynchronize and inter-communicate at predetermined timepoints after the initial synchronization to maintain the intended cooperation. For example, the controllers resynchronize every two minutes, every five minutes, every ten minutes, and/or the like, and inter-communication outside of these resynchronization timepoints is minimized.

With this example of “semi-coordinated” operation across controllers of modular chamber devices, a controller may be configured to detect operational errors or deviations, such as when the controller or a neighboring controller deviates from an inflation/deflation pattern. In some embodiments, a modular chamber device includes one or more sensors or sensing devices configured to detect an inflation state of a neighboring or adjacent modular chamber device. For example, referring again to FIGS. 8E, a modular chamber device can include a sensor 810 that is coupled, attached, or co-located with attachment mechanisms 804. The sensor 810 may include a strain sensor or strain gauge that measures forces exerted on the attachment mechanisms 804 as a result of the inflation or deflation of another modular chamber device connected to the attachment mechanisms 804. Accordingly, a modular chamber device is able to determine or estimate an inflation state of the other modular chamber device connected therewith, and based on the inflation state, determine whether the other modular chamber device is out-of-sync of a prescribed pattern. In some embodiments, an aggregate pressure-mitigation device is configured to optimize the creation of voids between multiple chambers based on each modular chamber device monitoring inflation and deflation of neighboring modular chamber devices. In some embodiments, if the controllers of an aggregate pressure-mitigation device detect that a particular modular chamber device has deviated from a pre-set inflation/deflation pattern, the controllers may re-synchronize with a particular controller of the particular modular chamber device.

FIG. 9B illustrates various inter-controller communications for one or more aggregate pressure-mitigation devices. In FIG. 9B, a first aggregate pressure-mitigation device 902A and a second aggregate pressure-mitigation device 902B are each composed of an arrangement of a plurality of modular chamber devices 800. Each modular chamber device 800 includes a controller 900. As discussed above, an aggregate pressure-mitigation device can include a central controller 900′ that is different than a controller 900 of a modular chamber device 800, and in the illustrated example, the first aggregate pressure-mitigation device 902A includes a central controller 900′. In the illustrated example, the first aggregate pressure-mitigation device 902A includes first controllers 900A collectively and the controllers of the first controllers 900A can communicate with one another and/or with the central controller 900′, and the second aggregate pressure-mitigation device 902B includes second controllers 900B collectively and the controllers of the second controllers 900B can communicate with one another and/or with a particular one of the second controllers 900B (e.g., a designated lead or master controller).

The first aggregate pressure-mitigation device 902A and the second aggregate pressure-mitigation device 902B can be deployed in separate location to apply respective pressure-mitigation treatments on different portions of a body. For example, the first aggregate pressure-mitigation device 902A is placed on a seat portion of a chair to provide a pressure-mitigation treatment on a sacral region of a body, while the second aggregate pressure-mitigation device 902B is placed on a back portion of the chair to provide a pressure-mitigation treatment on a lumbar and/or thoracic region of the body. In another example, the first aggregate pressure-mitigation device 902A is placed in a central portion of a bed over which a sacral region of a body is aligned, and the second aggregate pressure-mitigation device 902B is placed on a head or top portion of the bed over which a cranial region of the body is aligned. While the illustrated and described examples involve two aggregate pressure-mitigation devices, it is understood that any number of aggregate pressure-mitigation devices can be formed and deployed depending on the application.

To facilitate coordinated treatment for a body, multiple aggregate pressure-mitigation devices can communicate with one another, thereby forming another layer of communication above the communications between modular chamber devices of one aggregate pressure-mitigation device. For example, in FIG. 9B, the first controllers 900A and the second controllers 900B can communicate with one another to relay information regarding inflation cycles, synchronization times, errors/faults, and/or the like. Accordingly, in some embodiments, a controller of a modular chamber device and/or of an aggregate pressure-mitigation device communicates with another controller of another modular chamber device and/or of another aggregate pressure-mitigation device that is not necessarily connected to or arranged adjacent to the modular chamber device or the aggregate pressure-mitigation device.

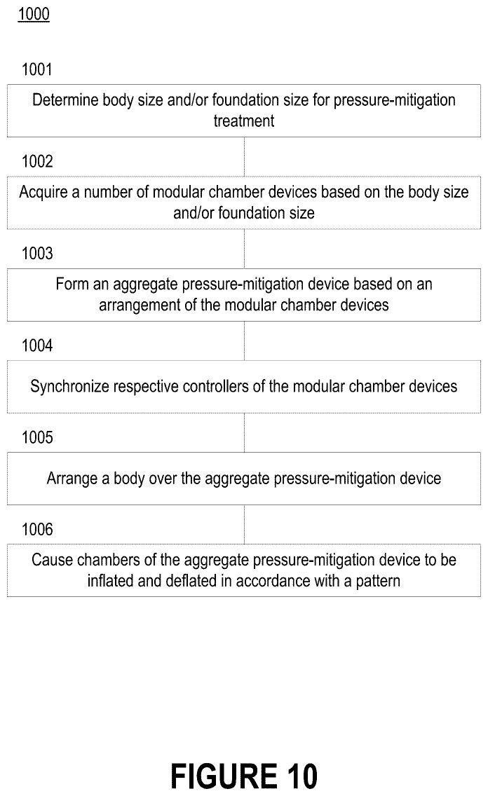

FIG. 10 is a flow diagram of a process 1000 for deploying a pressure-mitigation system designed to prevent and/or address ischemia-reperfusion injuries in accordance with embodiments of the present technology. The pressure-mitigation system can be composed of a modular arrangement of modular chamber devices that individually and separably operate respective inflatable chambers. The pressure-mitigation can then be adaptable (e.g., via the process 1000) to fit different body and/or foundation sizes via selective addition and/or removal of the modular chamber devices to and/or from the pressure-mitigation system.

Initially, an individual can determine a body size and/or a foundation size for a pressure-mitigation treatment (step 1001). For example, the individual can determine whether the body has a neonatal size, a pediatric size, an adult size, and/or the like. Similarly, the individual can determine the foundation or substrate on which the pressure-mitigation treatment will be provided. For example, the individual can determine that the body will be supported by a mattress, an operating table, a chair, a wheelchair, and/or the like. The individual can then acquire a number of modular chamber devices based on the determined body size and/or foundation size (step 1002). In particular, the individual acquires modular chamber devices that can be arranged to span the foundation size and/or the body size. Further, the individual can acquire modular chamber devices of particular shapes that can be arranged together to span the foundation size and/or the body size. In some embodiments, a set of modular chamber device shapes is associated with categories of body sizes and/or with foundation types. For example, a particular set of modular chamber devices is pre-determined for neonatal body sizes atop mattresses, while another set of modular chamber devices is pre-determined for adult body sizes atop wheelchairs. Thus, the individual can acquire modular chamber device according to pre-determined sets associated with the body size and/or the foundation size.

The individual can then form an aggregate pressure-mitigation device based on arranging and connecting the modular chamber devices (step 1003). The individual can arrange the modular chamber devices atop the foundation, and the modular chamber devices may span the foundation size based on the modular chamber devices being acquired based on the foundation size. The modular chamber devices can be arranged to form a geometric arrangement in which at least some of the edges of a modular chamber device are adjacent to edges of another modular chamber device. In some embodiments, arranging the modular chamber devices includes connecting the modular chamber devices to one another via attachment mechanisms.

The individual can then synchronize respective controllers of the modular chamber devices (step 1004). Synchronizing the respective controllers can include a controller identifying the other controllers included in the aggregate pressure-mitigation device, and initiating communications therewith. In some embodiments, synchronizing the controllers enables the controllers to begin independent operation of respective modular chamber devices in concert such that the aggregate pressure-mitigation device provides a pressure-mitigation treatment. In some embodiments, the individual can synchronize the controllers based on providing user input via a user interface of one or more of the controllers.

The individual can then arrange a body over the aggregate pressure-mitigation device (step 1005) for the body to be treated. In some examples, with the aggregate pressure-mitigation device being adapted for the body size, the body may be supported in entirety by the aggregate pressure-mitigation device. In some examples, the body may need to be oriented over a particular region (also referred to as a “target region”) of the aggregate pressure-mitigation device for the body to align with the body-adapted area of the aggregate pressure-mitigation device.

The individual can then cause chambers of the aggregate pressure-mitigation device to be inflated and deflated in accordance with a pattern (step 1006). In some embodiments, the individual operates the multiple controllers of the aggregate pressure-mitigation device in accordance with the synchronization and inter-communication between the multiple controllers. For example, the individual may operate one controller, which acts as the master or central controller and relays instructions to the other controllers. As another example, the multiple controllers are connected to a central external controller, and the individual may operate the central external controller. By operation of a controller by the individual, the pressure on anatomical region(s) of the human body can be varied via the controlled inflation, deflation, or combination thereof of the chambers of the aggregate pressure-mitigation device. The pattern may correspond to a configuration of the aggregate pressure-mitigation device, or in particular, a specific geometric arrangement and modular chamber devices that composes the aggregate pressure-mitigation device. For example, upon registering the multiple controllers of the aggregate pressure-mitigation device and identifying the chamber shapes of the aggregate pressure-mitigation device, one or more controllers may examine a library of patterns corresponding to different counts/arrangements of chambers to identify the appropriate pattern. Inflation (and deflation) of the chambers result in the respective heights of the chambers to dynamically change relative to other chambers and relative to the aggregate pressure-mitigation device.

Unless contrary to physical possibility, it is envisioned that the steps described above may be performed in various sequences and combinations. For example, the individual may arrange a body over the aggregate pressure-mitigation device prior to synchronizing the respective controllers of the modular chamber devices. By doing so, a weight of the body that may be sensed by the aggregate pressure-mitigation device can be used with the synchronization and inter-communication of the multiple controllers, for example, to determine whether only a subset of the modular chamber devices are needed to provide the pressure-mitigation treatment, to select a master or central controller of the multiple controllers, and/or the like. As another example, the respective controllers of the modular chamber devices may be synchronized prior to forming the aggregate pressure-mitigation device. By doing so, a synchronization and/or registration of the respective controllers to one another may enable one or more controllers to output instructions (e.g., auditory instructions, visual instructions) that instruct the individual how to arrange the modular chamber devices.

### Overview of Controller Devices

FIGS. 11A-11C are isometric, front, and back views, respectively, of a controller 1100 (also referred to as a “controller device”) that is responsible for controlling inflation and/or deflation of the chambers of pressure-mitigation devices in accordance with embodiments of the present technology. For example, the controller 1100 can be coupled to pressure-mitigation devices to control the pressure within the chambers of the pressure-mitigation devices. The controller 1100 can manage the pressure in each chamber of the pressure-mitigation devices by controllably driving one or more pumps. In some embodiments, a single pump is fluidically connected to all the chambers of the two or more pressure-mitigation devices, such that the pump is responsible for independently directing fluid flow to and/or from multiple chambers. In other embodiments, the controller 1100 is coupled to two or more pumps, each of which can be fluidically coupled to a single chamber to drive inflation/deflation of that chamber. In other embodiments, the controller 1100 is coupled to at least one pump that is fluidically coupled to two or more chambers and/or at least one pump that is fluidically coupled to a single chamber. The pump(s) may reside within the housing of the controller 1100 such that the system is easily transportable. Alternatively, the pump(s) may reside in a housing separate from the controller 1100.

As shown in FIGS. 11A-11C, the controller 1100 can include a housing 1102 in which internal components reside and a handle 1104 that is connected to the housing 1102. In some embodiments the handle 1104 is fixedly secured to the housing 1102 in a predetermined orientation, while in other embodiments the handle 1104 is pivotably secured to the housing 1102. For example, the handle 1104 may be rotatable about a hinge connected to the housing 1102 between multiple positions. The hinge may be one of a pair of hinges connected to the housing 1102 along opposing lateral sides. The handle 1104 enables the controller 1100 to be readily transported, for example, from a storage location to a deployment location (e.g., proximate a human body that is positioned on a surface). Moreover, the handle 1104 could be used to releasably attach the controller 1100 to a structure. For example, the handle 1104 could be hooked on an intravenous (IV) pole (also referred to as an “IV stand” or “infusion stand”).

In some embodiments, the controller 1100 includes a retention mechanism 1114 that is attached to, or integrated within, the housing 1102. Cords (e.g., electrical cords), tubes, and/or other elongated structures associated with the system can be wrapped around or otherwise supported by the retention mechanism 1114. Thus, the retention mechanism 1114 may provide strain relief and retention of an electrical cord (also referred to as a “power cord”). In some embodiments, the retention mechanism 1114 includes a flexible flange that can retain the plug of the electrical cord.

As further shown in FIGS. 11A-11C, the controller 1100 may include a connection mechanism 1112 that allows the housing 1102 to be securely, yet releasably, attached to a structure. Examples of structures include IV poles, mobile workstations (also referred to as “mobile carts”), bedframes, rails, handles (e.g., of wheelchairs), and tables. The connection mechanism 1112 may be used instead of, or in addition to, the handle 1104 for mounting the controller 1100 to the structure. In the illustrated embodiment, the connection mechanism 1112 is a mounting hook that allows for single-hand operation and is adjustable to allow for attachment to mounting surfaces with various thicknesses. In some embodiments, the controller 1100 includes an IV pole clamp 1116 that eases attachment of the controller 1100 to IV poles. The IV pole clamp 1116 may be designed to enable quick securement, and the IV pole clamp 1116 can be self-centering with the use of a single activation mechanism (e.g., knob or button).

In some embodiments, the housing 1102 includes one or more input components 1106 for providing instructions to the controller 1100. The input component(s) 1106 may include knobs (e.g., as shown in FIGS. 11A-11C), dials, buttons, levers, and/or other actuation mechanisms. An operator can interact with the input component(s) 1106 to alter the airflow provided to the two or more pressure-mitigation devices, discharge air from the pressure-mitigation device, or disconnect the controller 1100 from the two or more pressure-mitigation devices (e.g., by disconnecting the controller 1100 from tubing connected between the controller 1100 and the two or more pressure-mitigation devices).

As further discussed below, the controller 1100 can be configured to independently inflate and/or deflate one or more chambers of pressure-mitigation devices in a predetermined pattern specific for each pressure-mitigation device by managing one or more flows of fluid (e.g., air) produced by one or more pumps. In some embodiments the pump(s) reside in the housing 1102 of the controller 1100, while in other embodiments the controller 1100 is fluidically connected to the pump(s). For example, the housing 1102 may include a first fluid interface through which fluid is received from the pump(s) and a second fluid interface through which fluid is directed to the pressure-mitigation devices. Multi-channel tubing may be connected to either of these fluid interfaces. For example, multi-channel tubing may be connected between the first fluid interface of the controller 1100 and multiple pumps. As another example, multi-channel tubing may be connected between the second fluid interface of the controller 1100 and multiple valves of the pressure-mitigation devices. Here, the controller 1100 includes fluid interfaces 1108 designed to interface with multi-channel tubing. In some embodiments the multi-channel tubing permits unidirectional fluid flow, while in other embodiments the multi-channel tubing permits bidirectional fluid flow. Thus, fluid returning from the pressure-mitigation devices (e.g., as part of a discharge process) may travel back to the controller 1100 through the second fluid interface. By controlling the exhaust of fluid returning from the pressure-mitigation devices, the controller 1100 can actively manage the noise created during use.

By monitoring the connections with the fluid interfaces 1108, the controller 1100 may be able to detect which type of pressure-mitigation devices have been connected. Each type of pressure-mitigation device may include a different type of connector. For example, a pressure-mitigation device designed for elongated objects (e.g., the pressure-mitigation device 100 of FIGS. 1A-1B) may include a first arrangement of magnets in its connector, while a pressure-mitigation device designed for non-elongated objects may include a second arrangement of magnets in its connector. The controller 1100 may include one or more sensors arranged near the fluid interfaces 1108 that are able to detect whether magnets are located within a specified proximity. The controller 1100 may automatically determine, based on which magnets have been detected by the sensor(s), which types of pressure-mitigation devices are connected.

Pressure-mitigation devices may have different geometries, layouts, and/or dimensions suitable for various positions (e.g., supine, prone, sitting), various supporting objects (e.g., wheelchair, bed, recliner, surgical table), and/or various user characteristics (e.g., weight, size, ailment), and the controller 1100 can be configured to automatically detect the types of pressure-mitigation devices connected thereto. In some embodiments, the automatic detection is performed using other suitable identification mechanisms, such as the controller 1100 reading a radio-frequency identification (RFID) tag or barcode on the pressure-mitigation devices. Alternatively, the controller 1100 may permit an operator to specify the types of pressure-mitigation devices connected thereto. For example, the operator may be able to select, using an input component (e.g., input component 1106), a type of pressure-mitigation device via a display 1110. The controller 1100 can be configured to dynamically and independently alter the pattern for inflating and/or deflating chambers based on which types of pressure-mitigation devices are connected.

As shown in FIGS. 11A-11B, the controller 1100 may include a display 1110 for displaying information related to the pressure-mitigation devices, the pattern of inflations/deflations, the user, etc. For example, the display 1110 may present an interface that specifies which types of pressure-mitigation devices are connected to the controller 1100. As another example, the display 1110 may present an interface that specifies the programmable pattern that is presently governing inflation/deflation of the pressure-mitigation devices, as well as the current state within the programmable patterns for each pressure-mitigation device. Other display technologies could also be used to convey information to an operator of the controller 1100. In some embodiments, the controller 1100 includes a series of lights (e.g., light-emitting diodes) that are representative of different statuses to provide visual alerts to the operator or the user. For example, a status light may provide a green visual indication if the controller 1100 is presently providing therapy, a yellow visual indication if the controller 1100 has been paused (i.e., is in a pause mode), a red visual indication if the controller 1100 has experienced an issue (e.g., noncompliance of patient, patient not detected) or requires maintenance (i.e., is in an alert mode), etc. These visual indications may dim upon the conclusion of a specified period of time or upon determining that the status has changed (e.g., the pause mode is no longer active).

In some embodiments, the controller 1100 includes a rapid deflate function that allows an operator to rapidly and independently deflate pressure-mitigation devices. The rapid deflate function may be designed such that the entirety of a pressure-mitigation device is deflated or a portion (e.g., the side supports) of the pressure-mitigation device is deflated. This may be a software-implemented solution that can be activated via the display 1110 (e.g., when configured as a touch-enabled interface) and/or input components (e.g., tactile actuators such as buttons, switches, etc.) on the controller 1100. This rapid deflation, in particular the deflation of the side supports, is expected to be beneficial to operators when there is a need for quick access to the user, such as to provide cardiopulmonary resuscitation (CPR).

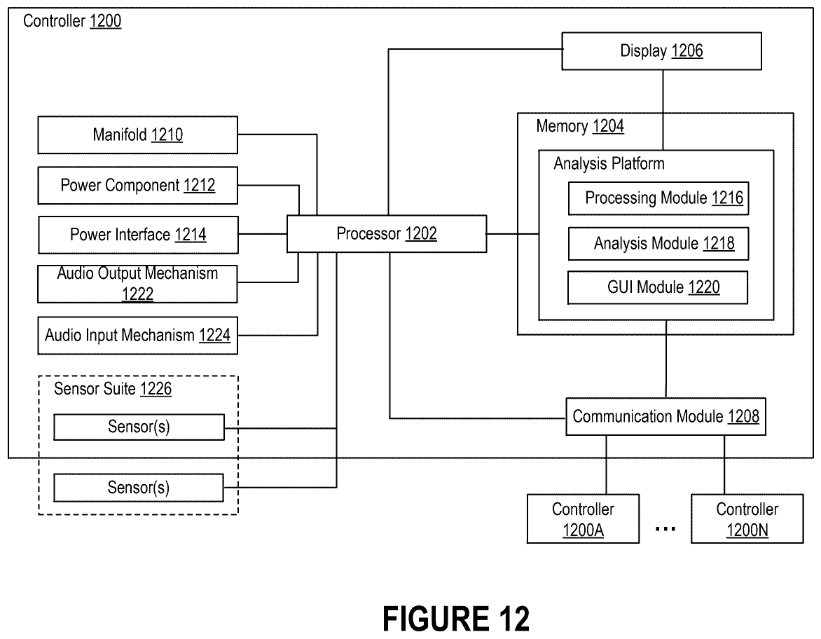

FIG. 12 illustrates an example of a controller 1200 in accordance with embodiments of the present technology. As shown in FIG. 12, the controller 1200 can include a processor 1202, memory 1204, display 1206, communication module 1208, manifold 1210, and/or power component 1212 that is electrically coupled to a power interface 1214. These components may reside within a housing (also referred to as a “structural body”), such as the housing 1102 described above with respect to FIGS. 11A-11C. In some embodiments, the aspects of the controller 1200 are incorporated into other components of a pressure-mitigation system. For example, some components of the controller 1200 may be incorporated into a computing device (e.g., a mobile phone or a mobile workstation) that is remotely coupled to two or more pressure-mitigation devices.

Each of these components is discussed in greater detail below. Those skilled in the art will recognize that different combinations of these components may be present depending on the nature of the controller 1200. Other components could also be included depending on the desired capabilities of the controller 1200.

For example, the controller could include one or more fragrance output mechanisms (e.g., spray pumps or spray nozzles) that are able to discharge scented fluid (e.g., air or liquid) from corresponding reservoirs, so as to produce an aroma. Such a feature may be desirable if one of the two or more pressure-mitigation devices is intended to be used as part of a therapy program.

As another example, the controller could include a circuitry that is able to detect and then examine electronic signatures emitted by nearby beacons. Accordingly, if an item (e.g., a wristband or file) that includes a beacon is presented to the controller, the controller may be able to detect the electronic signature emitted by the beacon and then take appropriate action. For instance, the controller may determine, based on the electronic signature that conveys information regarding the human body to be treated, how to independently inflate each of the chambers of the two or more pressure-mitigation devices. Electronic signatures may be transmitted via RFID, Bluetooth, NFC, or another short-range wireless communication protocol. Additionally or alternatively, the controller may be able to examine machine-readable codes (e.g., Quick Response codes, bar codes, and alphanumeric strings) that are printed on items such as wristbands, files, and the like. By examining the machine-readable code that is printed on an item associated with a human body, the controller may be able to determine, infer, or derive information regarding the human body. These features allow a controller to act as a “single action” solution for treating the human body since the controller may automatically begin treatment after an electronic signature or machine-readable code has been presented.

As discussed above, a controller can detect other controllers based on information communicated (e.g., wirelessly transmitted, passed along a wired connection) between the controller and other devices, including the other controllers or a lead/master/central controller. In some embodiments, a modular chamber device includes a beacon that is detectable by controllers of modular chamber devices, and a controller can detect other modular chamber devices in an aggregate pressure-mitigation device via respective beacons of the other modular chamber devices. In some embodiments, a controller of a modular chamber device includes an image sensor or a camera that can capture machine-readable information or human-readable information (e.g., Quick Response codes, alphanumerical identifiers) on a surface of another modular chamber device or controller thereof. Said information for another modular chamber device can indicate a shape of the other modular chamber device. Based on the detection of other controllers, a given controller can initiate a pairing process in which the given controller registers and identifies the other controllers.

The controller can also detect other controllers based on user input. For example, a user may indicate, via a user interface of a controller, a number of modular chamber devices (and controllers) are included in an aggregate pressure-mitigation device. In some embodiments, the controller stores in its memory a plurality of predefined geometric arrangements of modular chamber devices, and the user indicates, via a selection on a user interface from the plurality of predefined geometric arrangements, a particular predefined geometric arrangement in which the controller and its modular chamber device has been arranged. The user can further indicate a specific modular chamber device in the particular predefined geometric arrangement that corresponds to the controller and its modular chamber device.

In some embodiments, a user can define new geometric arrangements via a user interface of the controller and have the new geometric arrangements stored with the plurality of predefined geometric arrangements. For example, in response to determining that none of the predefined geometric arrangements are suitable for a particular application, the user arranges modular chamber devices in a new geometric arrangement and registers the new geometric arrangement in one or more controllers by inputting information that defines the new geometric arrangement. In some embodiments, the controller obtains the plurality of predefined geometric arrangements from an online database that is accessible by controllers of modular chamber devices, and the user can modify and add to the plurality of predefined geometric arrangements via the controller. For example, subsequent to receiving information related to a new geometric arrangement from a user, a controller uploads the information to the online database. In some examples, as discussed with FIG. 9B, a geometric arrangement can include adjacent modular chamber devices, separated modular chamber devices, modular chamber devices of different aggregate pressure-mitigation devices, and/or the like.

Based on a geometric arrangement selected by a user at a controller, the controller can detect a set of other controllers and initiate a control program that corresponds to the geometric arrangement for inflating and deflating the inflatable chambers. For example, based on a particular geometric arrangement including five modular chamber devices being identified, a given controller can search for and detect the four other controllers.

In some embodiments, the controller stores inflation program information in association with geometric arrangements. In particular, a geometric arrangement is associated with inflation program information that includes instructions for or describes inflation cycles for the modular chamber devices composing the geometric arrangement to provide pressure gradients and a pressure-mitigation treatment. The inflation program information can include time-wise information, such as timepoints for inflating an inflatable chamber, and spatial information, such as which inflatable chamber based on location within the geometric arrangement should be inflated at a given point in time. At least one controller of an aggregate pressure-mitigation device can retrieve the inflation program information based on the geometric arrangement of chambers within the aggregate pressure-mitigation device, and the at least one controller can relay instructions or commands to the other controllers to operate the aggregate pressure-mitigation device according to the inflation program information.

The processor 1202 can have generic characteristics similar to general-purpose processors, or the processor 1202 may be an application-specific integrated circuit (ASIC) that provides control functions to the controller 1200. As shown in FIG. 12, the processor 1202 can be coupled to all components of the controller 1200, either directly or indirectly, for communication purposes.

The memory 1204 may be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 1202, the memory 1204 can also store data generated by the processor 1202 (e.g., when executing the analysis platform). Note that the memory 1204 is merely an abstract representation of a storage environment. The memory 1204 could be comprised of actual memory chips or modules.

The display 1206 can be any mechanism that is operable to visually convey information to an operator. For example, the display 1206 may be a panel that includes LEDs, organic LEDs, liquid crystal elements, or electrophoretic elements. Alternatively, the display 1206 may simply be a series of lights (e.g., LEDs) that are able to indicate the status of the controller 1200. In some embodiments, the display 1206 is touch sensitive. Thus, an operator user may be able to provide input to the controller 1200 by interacting with the display 1206 itself. Additionally or alternatively, the operator may be able to provide input to the controller 1200 by interacting with input components, such as knobs, dials, buttons, levers, and/or other actuation mechanisms.

The communication module 1208 may be responsible for managing communications between the components of the controller 1200, or the communication module 1208 may be responsible for managing communications with other computing devices (e.g., a mobile phone associated with the operator, a network-accessible server system accessible to an entity responsible for manufacturing, providing, or managing pressure-mitigation devices). The communication module 1208 may be wireless communication circuitry that is designed to establish communication channels with other computing devices. Examples of wireless communication circuitry include integrated circuits (also referred to as “chips”) configured for Bluetooth®, Wi-Fi®, Near Field Communication (NFC), and the like.

Moreover, the communication module 1208 may be responsible for providing information for uploading to, and retrieving information from, the electronic health record that is associated with the human body that is presently being treated. Assume, for example, that the controller 1200 receives input indicating that a given person is to be treated using two or more pressure-mitigation devices. In such a situation, the controller 1200 may establish a connection with a storage medium that includes the electronic health record of the given person. In some embodiments the controller 1200 downloads information from the electronic health record into the memory 1204, while in other embodiments the controller 1200 simply accesses the information in the electronic health record. This information could be used to determine how to treat the given person. For example, the controller may determine, based on the weight and age of the given person, which patterns to select for inflating each of the chambers of the two or more pressure-mitigation devices, whether and when to adjust the patterns, etc.

The controller 1200 may be connected to pressure-mitigation devices that each includes a series of chambers whose pressure can be individually varied. When each pressure-mitigation device is placed between a human body and the surface of an object, the controller 1200 can independently cause the pressure on an anatomical region of the human body to be varied by controllably inflating and/or deflating chamber(s). Such action can be accomplished by the manifold 1210, which controls the flow of fluid to the series of chambers of each pressure-mitigation device.

Transducers mounted in the manifold 1210 can generate an electrical signal based on the pressure detected in each chamber of each pressure-mitigation device. Generally, each chamber is associated with a different fluid channel and a different transducer. Accordingly, if the manifold 1210 is designed to facilitate the flow of fluid to a pressure-mitigation device with four chambers, the manifold 1210 may include four fluid channels and four transducers. In some embodiments, the manifold 1210 includes fewer than four fluid channels and/or transducers or more than four fluid channels and/or transducers. Pressure data representative of the values of the electrical signals generated by the transducers can be stored, at least temporarily, in the memory 1204. The manifold 1210 may be driven based on a clock signal that is generated by a clock module (not shown). For example, the processor 1202 may be configured to generate signals for driving valves in the manifold 1210 (or driving integrated circuits in communication with the valves) based on a comparison of the clock signal to programmed patterns that indicate when each chamber of the two or more pressure-mitigation devices should be independently inflated or deflated. The programmed patterns may belong to a set of multiple programmed patterns that are stored in the memory 1204.

An analysis platform may be responsible for examining the pressure data. For convenience, the analysis platform is described as a computer program that resides in the memory 1204. However, the analysis platform could be comprised of software, firmware, or hardware that is implemented in, or accessible to, the controller 1200. In accordance with embodiments described herein, the analysis platform may include a processing module 1216, analysis module 1218, and graphical user interface (GUI) module 1220. Each of these modules can be an integral part of the analysis platform. Alternatively, these modules can be logically separate from the analysis platform but operate “alongside” it. Together, these modules enable the analysis platform to gain insights not only into whether the pressure-mitigation device connected to the controller 1200 is being used properly, but also into the health of the human body situation on or in the two or more pressure-mitigation devices.

The processing module 1216 can process pressure data obtained by the analysis platform into a format that is suitable for the other modules. For example, in preparation for analysis by the analysis module 1218, the processing module 1216 may apply algorithms designed for temporal aligning, artifact removal, and the like. Accordingly, the processing module 1216 may be responsible for ensuring that the pressure data is accessible to the other modules of the analysis platform. As further discussed below, the processor 1202 may forward at least some of the pressure data, in either its processed or unprocessed form, to the communication module 1208 for transmittal to a destination for analysis. In such a scenario, the processing module 1216 may apply operations (e.g., filtering, compressing, labelling) to the pressure data before it is forwarded to the communication module 1208 for transmission to the destination.

By examining the pressure data in conjunction with flow data representative of the fluid flowing into the controller 1200 from the pump(s), the analysis module 1218 can control how the chambers of the pressure-mitigation device are inflated and/or deflated. For example, the analysis module 1218 may be responsible for separately and independently controlling the set point for fluid flowing into each chamber such that the pressures of the chambers match a predetermined pattern for each pressure-mitigation device.

By examining the pressure data, the analysis module 1218 may also be able to sense movements of the human body under which each pressure-mitigation device is positioned. These movements may be caused by the user, another individual (e.g., a caregiver or an operator of the controller 1200), or the underlying surface. The analysis module 1218 may apply algorithms to the data representative of these movements (also referred to as “movement data” or “motion data”) to identify repetitive movements and/or random movements to better understand the health state of the user. For example, the analysis module 1218 may be able to produce a coverage metric indicative of the amount of time that the human body is properly positioned on or in each pressure-mitigation device. As further discussed below, the controller 1200 (or another computing device) may be able to independently establish whether each pressure-mitigation device has been properly deployed and/or operated based on the coverage metric. As another example, the analysis module 1218 may be able to establish the respiration rate, heart rate, or another vital measurement based on the movements of the user. Generally, the movement data are derived from the pressure data. That is, the analysis module 1218 may be able to infer movements of the human body by analyzing the pressure of the chambers of each of the pressure-mitigation devices in conjunction with the rate at which fluid is being delivered to those chambers. Consequently, some embodiments of each of the pressure-mitigation devices may not actually include any sensors for measuring movement, such as accelerometers, tilt sensors, or gyroscopes.

The analysis module 1218 may respond in several ways after examining the pressure data. For example, the analysis module 1218 may generate a notification (e.g., an alert) to be transmitted to another computing device by the communication module 1208. The other computing device may be associated with a medical professional (e.g., a physician or a nurse), a caregiver (e.g., a family member or friend of the user), or some other entity (e.g., a researcher or an insurer). As another example, the analysis module 1218 may cause the pressure data (or analyses of such data) to be integrated with the electronic health record of the user. Generally, the electronic health record is maintained in a storage medium that is accessible to the communication module 1208 across a network.

The GUI module 1220 may be responsible for generating interfaces that can be presented on the display 1206. Various types of information can be presented on these interfaces. For example, information that is calculated, derived, or otherwise obtained by the analysis module 1218 may be presented on an interface for display to the user or operator. As another example, visual feedback may be presented on an interface so as to indicate whether the user is properly situated on or in each pressure-mitigation device.

The controller 1200 may include a power component 1212 that is able to provide to the other components residing within the housing, as necessary. Examples of power components include rechargeable lithium-ion (Li-Ion) batteries, rechargeable nickel-metal hydride (NiMH) batteries, rechargeable nickel-cadmium (NiCad) batteries, etc. In some embodiments, the controller 1200 does not include a power component, and thus must receive power from an external source. In such embodiments, a cable designed to facilitate the transmission of power (e.g., via a physical connection of electrical contacts) may be connected between the power interface 1214 of the controller 1200 and the external source. The external source may be, for example, an alternating current (AC) power socket or another computing device. The cable connected to the power interface 1214 of the controller 1200 may also be able to convey power so as to recharge the power component 1212.

Embodiments of the controller 1200 can include any subset of the components shown in FIG. 12, as well as additional components not illustrated here.

For example, while the controller 1200 is able to receive and transmit data wirelessly via the communication module 1208, other embodiments of the controller 1200 may include a physical data interface through which data can be transmitted to another computing device. Examples of physical data interfaces include Ethernet ports, Universal Serial Bus (USB) ports, and proprietary ports.

As another example, some embodiments of the controller 1200 include an audio output mechanism 1222 and/or an audio input mechanism 1224. The audio output mechanism 1222 may be any apparatus that is able to convert electrical impulses into sound. One example of an audio output mechanism is a loudspeaker (or simply “speaker”). Meanwhile, the audio input mechanism 1224 may be any apparatus that is able to convert sound into electrical impulses. One example of an audio input mechanism is a microphone. Together, the audio output and input mechanisms 1222, 1224 may enable the user or operator to engage in an audible exchange with a person who is not located proximate the controller 1200. Assume, for example, that the user has become misaligned with one or more of the two or more pressure-mitigation devices. In such a scenario, the user may utilize the audio input mechanism 1224 to verbally ask for assistance, for example, from another person who is able to verbally confirm that assistance is forthcoming using the audio output mechanism 1222. The other person could be a medical professional or caretaker of the user. This may be useful in situations where the user is unable to reposition herself on or in one of the pressure-mitigation devices due to an underlying condition that inhibits or prevents movement.

The audio input mechanism 1224 may also be able to generate a signal that is indicative of more nuanced sounds. For example, the audio input mechanism 1224 may generate data that is representative of sounds originating from within the human body situated on or in one or more of the two or more pressure-mitigation devices. These sounds may be representative of auscultation sounds generated by the circulatory, respiratory, and gastrointestinal systems. These data could be transmitted (e.g., by the communication module 1208) to a destination for analysis.

Other sensors may also be implemented in, or accessible to, the controller 1200. For example, sensors may be contained in the housing of the controller 1200 and/or embedded within each pressure-mitigation device that is connected to the controller 1200. Collectively, these sensors may be referred to as the “sensor suite” 1226. For example, the sensor suite 1226 may include a motion sensor whose output is indicative of motion of the controller 1200 or each pressure-mitigation device. Examples of motion sensors include multi-axis accelerometers and gyroscopes. As another example, the sensor suite 1226 may include a proximity sensor whose output is indicative of proximity to the controller 1200 or pressure-mitigation device. A proximity sensor may include, for example, an emitter that is able to emit infrared (IR) light and a detector that is able to detect reflected IR light that is returned toward the proximity sensor. These types of proximity sensors are sometimes called laser imaging, detection, and ranging (LiDAR) scanners. Other examples of sensors include an ambient light sensor whose output is indicative of the amount of light in the ambient environment, a temperature sensor whose output is indicative of the temperature of the ambient environment, and a humidity sensor whose output is indicative of the humidity of the ambient environment. The output(s) produced by the sensor suite 1226 may provide greater insight into the environment in which the controller 1200 is deployed (and thus the environment in which the human body situated on or in each of the two or more pressure-mitigation devices is to be treated).

In some embodiments, the sensor suite 1226 includes one or more specialty sensors that are designed to generate, obtain, or otherwise produce information related to the health of the human body. For example, the sensor suite 1226 may include a vascular scanner. The term “vascular scanner” may be used to refer to an imaging instrument that includes (i) an emitter operable to emit electromagnetic radiation (e.g., in the near infrared range) into the body and (ii) a sensor operable to sense electromagnetic radiation reflected by physiological structures inside the human body. Normally, an image is created based on the reflected electromagnetic radiation that serves as a reference template for the vasculature of an anatomical region. Thus, the vasculature in an anatomical region could be periodically or continually monitored based on outputs produced by a vascular scanner included in the sensor suite 1226. Additionally or alternatively, the sensor suite 1226 may include sensors that are designed to perform pulse oximetry by determining oxygen level of the blood, measure blood pressure, compute heartrate, etc.

Based on the output(s) produced by the sensor suite 1226, the controller 1200 (or some other computing device) may be able to compute some or all of the main vital signs, namely, body temperature, blood pressure, pulse rate, and breathing rate (also referred to as “respiratory rate”). Moreover, the controller 1200 (or some other computing device) may be able to compute metrics that are indicative of the health of the human body, despite not being one of the main vital signs. For example, output(s) generated by the sensor suite 1226 could be used to establish whether the human body is performing a given activity (e.g., sleeping or eating). The output(s) could be used to not only ascertain the sleep pattern of the human body, but also whether changes in the sleep pattern indicate whether the health state of the human body has improved (e.g., sleep more consistent with longer duration following deployment of each pressure-mitigation device).

Note that the sensors included in the sensor suite 1226 need not necessarily be included in the controller 1200. For example, the controller 1200 may be communicatively connected to ancillary sensors that are included in various items (e.g., blankets and clothing), attached to the human body, etc.

These various components may allow the controller 1200 to be readily integrated into a network-connected environment, such as a home or hospital. Thus, the controller 1200 may be communicatively coupled to mobile phones, tablet computers, wearable electronic devices (e.g., fitness trackers and watches), or network-connected devices (also referred to as “smart devices”), such as televisions and home assistant devices. Similarly, the controller 1200 may be communicatively coupled to medical devices, such as cardiac pacemakers, insulin pumps, glucose monitoring devices, and the like. This level of integration can provide several notable benefits over conventional technologies for mitigating pressure.

As an example, the pressure-mitigation system of which the controller 1200 is a part may be used to monitor health of a human body in a more holistic sense. As mentioned above, insights into movements of the human body can be surfaced through analysis of pressure data generated by the controller 1200 or pressure-mitigation devices. Analysis of these movements over an extended period of time (e.g., days, weeks, or months) may lead to the discovery of abnormalities that might otherwise go unnoticed. For example, the controller 1200 (or some other computing device) may infer that the human body is suffering from an ailment in response to a determination that its movements over a recent interval of time differ from those that would be expected based on past intervals of time. At a high level, insights gained through analysis of the pressure data can be used not only to define a “health baseline” for the human body, but also to discover when deviations from the health baseline occur.

As another example, the controller 1200 may be responsible for providing or supplementing prompts to administer medication in accordance with a regimen. Assume, for example, that a user positioned on or in one or more of the two or more pressure-mitigation devices is associated with a regimen that requires a medication be administered regularly. The controller 1200 may promote adherence to the regimen by prompting the user or another person (e.g., an operator of the controller 1200) to administer the medication. Visual notifications could be presented by the display 1206, or audible notifications could be presented by the audio output mechanism 1222. Additionally or alternatively, the controller 1200 could cause digital notifications (also referred to as “electronic notifications”) to be presented by a computing device that is communicatively coupled to the controller 1200. In some embodiments, the regimen is stored in the memory 1204 of the controller 1200. In other embodiments, the regimen is stored in the memory of a computing device that is communicatively coupled to the controller 1200. For example, the regimen may be implemented by a computer program that is executing on a mobile device associated with the user, and when the computer program determines that a dose of the medication is due to be administered, the computer program may transmit an instruction to the controller 1200 to generate a notification.

As another example, the controller 1200 may be able to facilitate communication with medical professionals. Assume, for example, that the controller 1200 is deployed in a home environment that medical professionals visit infrequently or not at all. In such a scenario, the controller 1200 may allow the user to communicate with medical professionals who are located outside of the home environment. Thus, the user may be able to communicate, via the audio output and input mechanisms 1222, 1224, with medical professionals who are located in a hospital environment (e.g., at which the user received treatment) or their own home environments.

As another example, the controller 1200 may be able to facilitate communication with emergency services. For instance, if the controller 1200 determines (e.g., through analysis of pressure data) that no movement has occurred for a predetermined amount of time, the controller 1200 may prompt the user to respond. Similarly, if the controller 1200 receives input from the user indicative of a request for assistance, the controller 1200 may initiate communication with emergency services. Thus, the controller 1200 may be programmed to person some action if, for example, it determines (e.g., through analysis of the signal generated by the audio input mechanism 1224) that the user has indicated she has fallen or has experienced a medical event (e.g., shortness of breath, heart palpitations, excessing sweating).

These benefits allow pressure-mitigation systems to be deployed in situations where frequent visits by medical professionals may not be practical or possible. For example, when deployed in a hospital environment, a pressure-mitigation system may allow medical professionals to visit patients less frequently. Patients situated on or in two or more pressure-mitigation devices may not need to be turned to alleviate pressure as often, and medical professionals may not need to continually check on patients if pressure-mitigation systems are able to autonomously discover changes in health. As another example, when deployed in a home environment, a pressure-mitigation system may be able to counter a lack of visits from medical professionals. If a patient is instructed to situate herself on or in one or more of two or more pressure-mitigation devices while at home, the patient may only need to be visited every few (e.g., three, five, or seven) days rather than once per day or multiple times per day. Overall, implementing pressure-mitigation systems can lead to significant cost savings because medical professionals are required to make less frequent visits and perform fewer medical procedures, and because patients can be discharged more quickly.

The controller 1200 may also be designed to focus on wellness in addition to, or instead of, treatment for (and prevention of) pressure-induced injuries. As an example, embodiments of the controller 1200 may be designed to aid in sleep management, for healthy individuals and/or unhealthy individuals. Using the audio output mechanism 1222 in combination with the manifold 1210, the controller 1200 may be able to accomplish tasks such as simulating the presence of another person, for example, by producing vocal sounds, breathing sounds, applying pressure, and the like.

### Processing System

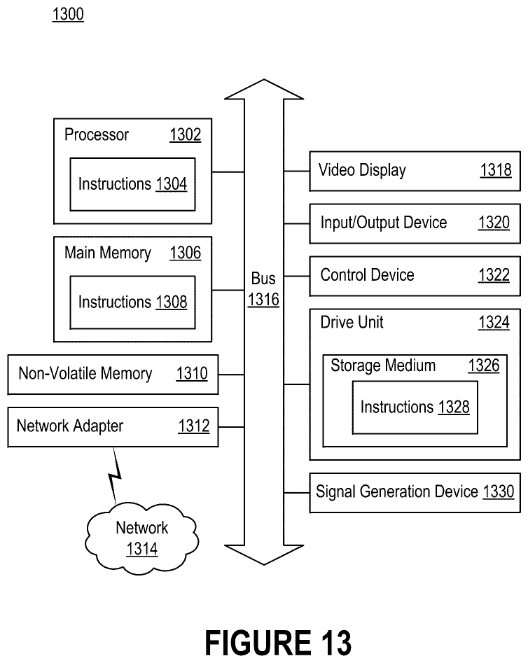

FIG. 13 is a block diagram illustrating an example of a processing system 1300 in which at least some operations described herein can be implemented. For example, components of the processing system 1300 may be hosted on a controller responsible for controlling the flow of fluid to each pressure-mitigation device. As another example, components of the processing system 1300 may be hosted on a computing device that is communicatively coupled to the controller.

The processing system 1300 may include a processor 1302, main memory 1306, non-volatile memory 1310, network adapter 1312 (e.g., a network interface), video display 1318, input/output device 1320, control device 1322 (e.g., a keyboard, pointing device, or mechanical input such as a button), drive unit 1324 that includes a storage medium 1326, or signal generation device 1330 that are communicatively connected to a bus 1316. The bus 1316 is illustrated as an abstraction that represents one or more physical buses and/or point-to-point connections that are connected by appropriate bridges, adapters, or controllers. The bus 1316, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or bus compliant with Institute of Electrical and Electronics Engineers (IEEE) Standard 1394.

The processing system 1300 may share a similar computer processor architecture as that of a computer server, router, desktop computer, tablet computer, mobile phone, video game console, wearable electronic device (e.g., a watch or fitness tracker), network-connected (“smart”) device (e.g., a television or home assistant device), augmented or virtual reality system (e.g., a head-mounted display), or another computing device capable of executing a set of instructions (sequential or otherwise) that specify action(s) to be taken by the processing system 1300.

While the main memory 1306, non-volatile memory 1310, and storage medium 1326 are shown to be a single medium, the terms “storage medium” and “machine-readable medium” should be taken to include a single medium or multiple media that stores one or more sets of instructions 1328. The terms “storage medium” and “machine-readable medium” should also be taken to include any medium that is capable of storing, encoding, or carrying a set of instructions for execution by the processing system 1300.

In general, the routines executed to implement the embodiments of the present disclosure may be implemented as part of an operating system or a specific application, component, program, object, module, or sequence of instructions (collectively referred to as “computer programs”). The computer programs typically comprise one or more instructions (e.g., instructions 1304, 1308, 1328) set at various times in various memories and storage devices in a computing device. When read and executed by the processor 1302, the instructions cause the processing system 1300 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 1310, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 1312 enables the processing system 1300 to mediate data in a network 1314 with an entity that is external to the processing system 1300 through any communication protocol supported by the processing system 1300 and the external entity. The network adapter 1312 can include a network adaptor card, a wireless network interface card, a switch, a protocol converter, a gateway, a bridge, a hub, a receiver, a repeater, or a transceiver that includes an integrated circuit (e.g., enabling communication over Bluetooth or Wi-Fi).

The techniques introduced here can be implemented using software, firmware, hardware, or a combination of such forms. For example, aspects of the present disclosure may be implemented using special-purpose hardwired (i.e., non-programmable) circuitry in the form of application-specific integrated circuits (ASICs), programmable logic devices (PLDs), field-programmable gate arrays (FPGAs), and the like.

### Remarks

The foregoing description of various embodiments has been provided for the purposes of illustration and description. It is not intended to be exhaustive or to limit the disclosed subject matter to the precise forms disclosed. Many modifications and variations will be apparent to one skilled in the art. Embodiments were chosen and described in order to best describe the principles of the invention and its practical applications, thereby enabling those skilled in the relevant art to understand the disclosed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the patent document has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

1. A pressure-mitigation system comprising:
a first modular chamber device including:
a first inflatable chamber in the form of a first geometric shape, and
a first controller configured to control inflation of the first inflatable chamber; and
a second modular chamber device including:
a second inflatable chamber in the form of a second geometric shape that is able to fit adjacent to the first geometric shape,
a second controller configured to control inflation of the second inflatable chamber, and
an attachment mechanism coupled to the second inflatable chamber and configured to connect the second inflatable chamber with the first inflatable chamber;
wherein the first and second inflatable chambers are adjacently connected via the attachment mechanism in a geometric arrangement, such that the geometric arrangement of the first and second modular chamber devices provides a continuous surface configured to support a human body disposed thereon; and
wherein the first controller communicates with the second controller to apply a pressure-mitigation treatment on the human body based on a coordination of the inflation of the first inflatable chamber and the inflation of the second inflatable chamber that results in a pressure differential being applied by the first and second inflatable chambers on the human body.

2. The pressure-mitigation system of claim 1, further comprising:
a central controller to which the first controller and the second controller are communicably coupled, wherein the central controller is configured to transmit commands to each of the first controller and the second controller to facilitate the coordination of the inflation of the first inflatable chamber and the inflation of the second inflatable chamber.

3. The pressure-mitigation system of claim 1, wherein the first modular chamber device further includes a respective attachment mechanism configured to interface with the attachment mechanism of the second modular chamber device.

4. The pressure-mitigation system of claim 1, wherein the attachment mechanism of the second modular chamber device is located on an externally-facing edge of the second geometric shape of the second inflatable chamber.

5. An aggregate pressure-mitigation device comprising:
a plurality of modular chamber devices located in a geometric arrangement, each modular chamber device including:
an inflatable chamber, and
a controller configured to control an inflation of the inflatable chamber;
wherein the respective controllers of the modular chamber devices are configured to communicate with one another to coordinate the inflation of the respective inflatable chambers according to a pattern to effect a pressure-mitigation treatment on a body disposed atop the aggregate pressure-mitigation device.

6. The aggregate pressure-mitigation device of claim 5, wherein each modular chamber device further includes an attachment mechanism configured to adjacently connect a respective modular chamber device to another modular chamber device in the geometric arrangement.

7. The aggregate pressure-mitigation device of claim 5, wherein the respective controllers of the modular chamber devices are configured to coordinate the inflation of the respective inflatable chambers via a central controller to which each of the respective controllers are communicably coupled.

8. The aggregate pressure-mitigation device of claim 5, wherein the respective controllers coordinate the inflation of the respective inflatable chambers such that a number of occurrences in which inflatable chambers that belong to adjacent modular chamber devices in the geometric arrangement are concurrently inflated in a way that precludes a void being created between the adjacent modular chamber devices.

9. The aggregate pressure-mitigation device of claim 5, wherein a modular chamber device is configured to be detached from the geometric arrangement, such that a surface provided by the aggregate pressure-mitigation device via the geometric arrangement is reduced in size.

10. The aggregate pressure-mitigation device of claim 5, wherein at least one of the plurality of modular chamber devices include an inflatable chamber configured with a different geometric shape than that of another modular chamber device.

11. The aggregate pressure-mitigation device of claim 5, wherein the plurality of modular chamber devices lacks intervening tubing between each of the modular chamber devices.

12. A method of operating a pressure-mitigation system, the method comprising:
determining a body size of a subject of a pressure-mitigation treatment;
acquiring a set of modular chamber devices that is associated with the body size, wherein the set includes modular chamber devices having one or more different geometric shapes, and wherein a total area spanned by the modular chamber devices having the one or more different geometric shapes corresponds to the body size;
forming an aggregate pressure-mitigation device based on positioning the set of modular chamber devices in a geometric arrangement;
disposing the body over the aggregate pressure-mitigation device; and
causing the aggregate pressure-mitigation device to be operated to provide the pressure-mitigation treatment.

13. The method of claim 12, wherein each modular chamber device includes a respective controller, and wherein causing the aggregate pressure-mitigation device to be operated includes:
synchronizing the respective controllers of the set of modular chamber devices with one another; and
causing the respective controllers to communicate with one another such that the respective controllers individually operate respective modular chamber devices in concert to provide the pressure-mitigation treatment.

14. The method of claim 12, wherein each modular chamber device includes a respective controller, and wherein causing the aggregate pressure-mitigation device to be operated includes:
communicatively coupling the respective controllers of the set of modular chamber devices to a central controller such that each respective controller is able to receive commands from the central controller for operating the aggregate pressure-mitigation device.

15. The method of claim 12, wherein the set of modular chamber devices includes at least one controller, and wherein the method further comprising:
inputting, to the at least one controller, an indication of the geometric arrangement of the modular chamber devices.

16. The method of claim 12, wherein each of the set of modular chamber devices comprises an inflatable chamber in the form of a respective geometric shape.

17. The method of claim 16, wherein each of the set of modular chamber devices comprises a first attachment mechanism located at a first edge of the respective geometric shape.

18. The method of claim 17, wherein a modular chamber device of the set of modular chamber devices comprises a second attachment mechanism located at a second edge of the respective geometric shape, the second attachment mechanism having a different detachment threshold than the first attachment mechanism.

19. The method of claim 12, wherein the set of modular chamber devices is acquired using a mapped backing that comprises indications that visually convey the geometric arrangement.

20. The method of claim 19, wherein the mapped backing further comprises raised ridges that enable the set of modular chamber devices to rest in the geometric arrangement.
